(19) United States
(12) Patent Application Publication
(10) Pub. No.: US 2021/0213300 A1
(43) Pub. Date: Jul. 15, 2021

(54) Methods of Treating Neuropsychiatric Disorders

(71) Applicant: ROBERT J. HEDAYA, BETHESDA, MD (US)

(72) Inventor: ROBERT J. HEDAYA, BETHESDA, MD (US)

(21) Appl. No.: 17/079,876

(22) Filed: Oct. 26, 2020

Related U.S. Application Data
(60) Provisional application No. 62/960,879, filed on Jan. 14, 2020.

(51) Int. Cl. A61N 5/06

(52) U.S. Cl. CPC A61N 5/0618; A61N 2005/067

(57) ABSTRACT

The claimed invention includes methods of treatment using laser radiation to treat one or more brain abnormalities. The methods can include assessing a subject's condition to identify a condition and/or tissue in a subject in need of treatment and administering laser radiation to a subject. In some embodiments, the parameters of the laser treatment are adjusted to a specific response in a subject in need thereof.

This application claims priority to U.S. Provisional Patent Application No. 62/960,879, filed on Jan. 14, 2020 and now pending, the entirety of which is incorporated by reference.

### BACKGROUND OF THE PRESENT INVENTION

Photo-biomodulation (PBM) is the application of red or near infrared light to tissues in order to stimulate, heal, and/or protect tissue that has been damaged, is degenerating, and/or at risk of dying. PBM may also be used to enhance the function of healthy tissue. Because the brain can suffer from degeneration and damage thorough various insults (e.g., stroke, mechanical or emotional trauma, nutritional deficits, toxins, hormonal imbalances, inflammation, oxygen deprivation, aging, genetic vulnerabilities, and epigenetic dysfunction), physicians and scientists have applied light emitting diodes (LEDs) and lasers to the brain to try to heal it. The current focus in the field has moved away from lasers as a light source and now emphasizes low-level light therapy [LLLT]. Some opinions, as reported by Hamblin—suggest that a laser is not necessarily needed. (Hamblin M R. Shining light on the head: Photobiomodulation for brain disorders. BBA Clin. 2016; 6:113-124. Published 2016 Oct. 1. doi:10.1016/j.bbacli.2016.09.002).

Moreover, methods associated with applying light to tissues described in the prior art focus on light parameters such as wavelength, power density, energy density, and total energy but indiscriminately irradiate a subject's brain using a light source with a hope of a beneficial effect much akin to chemotherapy in its indiscriminate field of action. Further, current use of light to treat the brain does not assess the nature and needs of the receiving tissue when making the determination of application parameters. Accordingly, there is a need for a method of treating a subject that can specifically direct beneficial neural change at defined locations, and with tissue specific parameters of a subject's brain, based on objective data, to identify and treat the root neural dysfunctions of neuropsychiatric disorders.

### SUMMARY OF THE INVENTION

In some embodiments, the present invention is a method of treating a neuropsychiatric or physical disorder in a subject or patient that includes functional treatment of the subject; including analyzing brain activity of the subject; determining a treatment location or locations for application of a laser; selecting laser parameters; administering a laser at or for the treatment location(s); repeating the analysis of the subjects brain activity and based on changes in said brain activity, modifying the laser treatment parameters. While stated here as a laser, alternate light emitting devices, such as but not limited to LEDs may be used in some circumstances. The waveform delivered may be continuous, pulsed, or some combination and may differ by condition.

The present invention is directed to the combination of photobiomodulation therapy (PBMT), using a laser or an LED as examples, in conjunction with quantitative electroencephalogram (qEEG), or equivalent, for treating a patient for any of a variety of neurological conditions and/or to enhance specific brain performance. In at least one embodiment, magnetic resonance imaging of the brain and/or other portions of the patient's anatomy are also used as input. In particular, a qEEG is used, at least in part, for identifying specific areas in the brain where functioning is outside a normal range and therefore determining parameters associated with application of the laser therapy to a patient. In short, the qEEG (or equivalent) result is used as an input to identifying the location of preferred application in the patient of laser therapy as well as laser parameters (pulse frequency, quantity of treatments, frequency of treatments, etc.). A qEEG ordinarily is a diagnostic tool that measures electrical activity in the form of brain wave patterns. It is sometimes referred to as “brain mapping.” The qEEG is used in the present invention in a somewhat different way—to identify relevant nerve tracts and cortical Brodmann Areas (BAs) for applying subsequent laser therapy to those tracts and/or BAs in a manner consistent with optimizing or improving the function of tracts or BAs.

In a preferred embodiment, the present invention is computer driven and is controlled and/or operated by a processor, where the processor is in communication with a laser system, presumably with a plurality of selectable lasers, and a testing system for testing the patient. In additional, the processor of the present invention has the ability to accept data for determining diagnoses and making decisions for additional steps based on the diagnoses.

The method(s) of the present invention can include using an Fx-Hylane program in healthy individuals to augment performance. The Fx-Hylane program is comprised of two parts: “Fx” and “Hylane”. Fx represents Functional Medicine (Fx). Functional medicine is a field of medicine in which chronic medical conditions are evaluated through a systems biology lens. Specifically, root causes of a condition are identified (via history, physical exam, and testing, etc.). There are three pillars of Functional Medicine. First, antecedents (factors which increased vulnerability to an illness), triggers (factors which may have activated an illness), and mediators (factors that maintain the disease process) of the condition are identified. Second various systems (e.g., mental, emotional, spiritual, assimilation, defense, and repair; energy, biotransformation, and elimination; transport; communication; and structural integrity) are assessed for dysfunction. Third, modifiable lifestyle factors (including but not limited to sleep/restoration, exercise, nutrition, stress, and relationships) are assessed to determine where adjustment is needed. Once the data (history, physical, laboratory testing) is obtained it is analyzed to determine abnormalities and interventions. Once the Fx part of the program is incorporated into the treatment, the Hylane treatment may be added in. Hylane refers to Hyperbaric Oxygen Therapy (Hy), Laser (la), neuronal exercises (ne). The Hylane arm of treatment is based on history, neuropsychological testing, physical exam and analysis of at least one quantitative electroencephalogram (qEEG) or equivalent. These data determine which aspects of the Hylane program in addition to the laser (light emitting devices are collectively referred to as lasers herein, which is always included) would be useful for the patient. In some embodiments, a desired behavioral outcome is selected, typically in advance (e.g., improving working memory, faster and/or more accurate discrimination of targets embedded in a background field, improving psychomotor reaction time, improving resilience to stress, and/or reducing perceived stress levels of multivariate tasks) and the Fx-HyLane program is applied after baseline measures are taken. In some embodiments, the application of the Fx-Hylane program is sequentially or concurrently paired with specified exercises, such as neurofeedback, that challenge the specific circuits and pathways which serve the desired behavioral outcome. Following application of the Fx-Hylane program, repeated performance measures may be taken.

### BRIEF DESCRIPTION OF THE FIGURES

General Key to understanding the figures:

Areas of over activity (yellow/orange) or underactivity (light blue/dark blue) on the surface of the brain or deep in the brain are indicated by color. The figures depict information about several neuronal processes:
a) The surface of the brain (the cortex). When the surface amplitude (in microAmps) at a specific cortical location or Brodmann Area (BA) is normal (+ or −Z-score of 2.0. or less, i.e., within 2 standard deviations from the mean), it is gray; When the surface amplitude at a specific Brodmann Area (BA) in a specific neuronal frequency (e.g. 1-4 Hz, delta) is underactive in amplitude (Z score below −2.0 SD) it is light blue (mild) or dark blue (severe); When the surface amplitude at a specific BA in a specific a specific neuronal frequency (e.g. 1-4 Hz, delta) is excessive in amplitude (Z score greater than 2.0) it is yellow/orange (mild) or red (severe).
b) Coherence of connections between different surface Brodmann Areas (BA) regions. Coherence is a term that quantifies the frequency and amplitude of the synchronicity of neuronal patterns of oscillating neuronal activity. It is a measure of whether groups of neurons are firing in phase (normal), excessively in phase (hypercoherent, or hyper-linked in their firing pattern), or excessively out of phase (hypocoherent, or decreased linkage in their firing pattern). Normal phase coherence does not show up in the images; excessive in-phase coherence (hypercoherence) shows up as yellow (mild) or red (severe) tubular lines. Excessive out-of-phase coherence (hypocoherence) is illustrated as blue (mild) or dark blue (severe) tubular lines.
c) DTI (Diffuse Tensor Imaging): This is a calculation which approximates the microstructural dysfunction in sub-cortical (i.e., deep) white matter tracts detected on magnetic resonance imaging. Based on evidence (Scrascia, Federica et al. ‘Relationship Among Diffusion Tensor Imaging, EEG Activity, and Cognitive Status in Mild Cognitive Impairment and Alzheimer's Disease Patients’. 1 Jan. 2014: 939-950.) of convergence among EEG rhythm changes, and DTI values (as determined by magnetic resonance) dysfunction in these subcortical deep white matter tracts is identified as underactive (light blue/dark blue), or overactive (yellow/red) thin lines.
d) The cross hairs (red) are focused on the area of maximal Z-score deviation in that particular Hz (frequency) within the brain.
e) The panel with the red-dotted circle is a representation of the different surface Brodmann areas (the red dots) and the degree of coherence between them (the thin colored lines between the different BA's). Hypercoherence is reflected as yellow/orange/red, and hypocoherence is reflected as blue/dark blue. Normalization of coherence between BA's is reflected by the absence of any connecting line (i.e., blackness).
f) The panel to the far right in the figures reflects the BA's and their degree of deviation (Z-score) from the mean. Deviation greater than 1.65-2.0 SD (depending on the setting) is reflected by pink coloration.

In general and to summarize, normalization is determined by using light therapy to reduce or eliminate areas of over activity (yellow/orange) or underactivity (light blue/dark blue). On the surface of the brain normalization is reflected by gray color. Network dysfunction is indicated by yellow/orange/red and light/dark blue. The neuronal connections with the most disturbed function are red (excess) and dark blue (under). A more mild disturbance in function is reflected by yellow (excess) and light blue (under). When network function between brain areas is normalized there will be a reduction of the number of red/yellow/blue lines. Complete normalization of network connectivity is manifest by a lack of any red/orange/yellow/blue lines.

FIGS. 1-3 display qEEG results for patient PM.

FIG. 1 shows the baseline (pre-laser, post HYLANE) qEEG on Jan. 19, 2019.

FIG. 2 depicts figure shows the eyes open condition at 6 Hz on Mar. 19, 2020, after 21 laser treatments.

FIG. 3, from Apr. 22, 2019, in the eyes open condition @ 6 Hz, demonstrates continued improvement 21 days after the last laser treatment. This is demonstrated by the lower Center Values in the different Brodmann Areas.

FIGS. 4-7 display follow up qEEG results for patient JL.

FIG. 4: At 24/25 Hz: Change between treatments #10 (Left) and #20 (right)—Many areas of DTI abnormality (blue) on patients right (left side of image) are gone;

FIG. 5: Baseline (left) and after 20 laser treatments 26 Hz—areas of hypo-coherence nearly gone.

FIG. 6 depicts the DTI (diffuse tensor imaging) in the right and left hemispheres of the brain @ 26 Hz in the mood/depression network.

FIG. 7 depicts the DTI (diffuse tensor imaging) in the right and left hemispheres of the brain @ 26 Hz in the working memory network.

FIGS. 8-11 display follow up qEEG results for patient AK.

FIG. 8: depicts 18 Hz: Left Inferior Fronto Occipital Fasciculus—Temporal Connection—Visual Object Recognition, Semantic Processing.

FIG. 9 depicts 17 Hz Pre (Right) Post (Left) Vertical Occipital Fasciculus (Vision and cognition, and reading).

FIG. 10 depicts pre (Left panel)—Post (right panel) qEEG: Parieto-pontine tract normalized.

FIG. 11 depicts normalization of Left Inferior Fronto-Occipital Fasciculus: Salience network, semantic language.

FIG. 12 depicts a simplified schematic diagram of the present invention.

### DETAILED DESCRIPTION OF THE INVENTION

Before the present compositions and methods are described, it is to be understood that this invention is not limited to the particular processes, compositions, or methodologies described, as these may vary, such as from patient to patient. It is also to be understood that the terminology used in the description is for the purpose of describing the particular versions or embodiments only and is not intended to limit the scope of the present invention, in that permutations or combinations of the embodiments described herein could be employed, as could reasonable variations thereof. Unless defined otherwise, all technical and scientific terms used herein have the same meaning as commonly understood by one of ordinary skill in the art. Although any methods and materials similar or equivalent to those described herein can be used in the practice or testing of embodiments of the present invention, the preferred methods, devices, and materials are now described. All publications, literature, hyperlinks mentioned or cited herein are incorporated by reference in their entirety. Nothing herein is to be construed as an admission that the invention is not entitled to antedate such disclosure(s) by virtue of prior invention.

It must also be noted that as used herein and in the appended claims, the singular forms “a,” “an,” and “the” include plural reference unless the context clearly dictates otherwise. Thus, for example, reference to a “cell” is a reference to one or more cells and equivalents thereof known to those skilled in the art, and so forth.

As used herein, “administering,” when used in conjunction with a therapeutic, means to administer a therapeutic directly to a subject.

The terms “treat,” “treated,” or “treating” as used herein refer to therapeutic treatment and/or prophylactic or preventative measures, wherein the object is to prevent slow down (lessen), or eliminate an undesired physiological condition, disorder, or disease, or to obtain beneficial or desired clinical results. For the purposes of this invention, beneficial or desired results include, but are not limited to, alleviation of symptoms; diminishment of the extent of the condition, disorder, or disease; stabilization (i.e., not worsening) of the state of the condition, disorder, or disease; delay in onset or slowing of the progression of the condition, disorder, or disease; amelioration of the condition, disorder, or disease state; and remission (whether partial or total), whether detectable or undetectable, or enhancement or improvement of the condition, disorder, or disease; enhancement of specific functions in healthy individuals.

The term “subject,” as used herein, describes an organism, including mammals, to which treatment with the compositions and compounds according to the subject disclosure can be administered. Mammalian species that can benefit from the disclosed methods include, but are not limited to, apes, chimpanzees, orangutans, humans, monkeys; and other animals such as dogs, cats, horses, cattle, pigs, sheep, goats, chickens, mice, rats, guinea pigs, and hamsters. Typically, the subject is a human.

Part of the present invention may involve determinative aspects related to the specific patient. That is, the process may determine the best location and type of laser application, and the frequency and duration of the applications. Determination of success may be involved after each of or after several laser treatments.

A goal of the present invention is to enhance or improve a patient's functioning, such as but not limited to cognitive functioning.

In addition, a target population for this process is patients who typically had generally positive neurological conditions which deteriorated at some point or over time, such as from accidents, or diagnoses, or conditions viewed by the patient or a caregiver as out of the norm. That is, candidates for such a process are those who have neuro-degraded conditions which have potential for improvement and patients who have specific functions needing enhancement, such as improved focus or improved cognition. The present invention is suited to such patients as it is directed to targeted pathways and targeted conditions and methodologies.

While not wishing to be bound by theory, an embodiment of the present invention includes use of light, preferably a laser, which enters a tissue at a point of focus, which is thought to work by several mechanisms. For example, one mechanism includes photon induced dissociation of nitric oxide (NO) from complex four (cytochrome C oxidase, or CCO) of the mitochondrial respiratory chain. This dissociation allows the release and production of increased amounts of ATP (adenosine triphosphate), which is the energy molecule that drives all physiological processes. The NO released is also believed to increase blood flow to the irradiated area of the tissue, resulting in greater oxygen availability. Further, an increase in reactive oxygen species (ROS) also activates genetic cell-protection. Activation of cellular transcription factors that result in long term changes in cellular structure and function are also attributed to irradiation of tissue by certain light sources such as a laser. Accordingly, the photon induced increase in energy (ATP) and blood flow (oxygen), and the placement of cognitive demand on the weak neural area, or the neural networks whose performance is to be enhanced, will cause the neurons to use the energy for the function and development of the deficient and/or performance targeted neurons.

As discussed above, current methods of treatment in the prior art disregard evaluating the needs of the treated tissue, whether it be to determine specific locations to treat within the tissue, the nature of the problem, and the proper parameters for treatment, such as a determination of which specific neuronal populations require attention. Accordingly, embodiments of the present invention assess the location, nature, and needs of the receiving tissues (e.g., excess or deficient amplitude in specific neuronal frequencies, excess or reduced coherence [connectivity], patterns of information flow, phase lag, network dysregulations, current source densities, assessment of all cortical Broadmann Areas, inter and intra-hemispheric connectivity, etc.) and adjusts application parameters accordingly.

Other approaches to treating brain-based disorders that are not laser based include approaches such as magnetic pulsation and various types of electrical stimulation. The laser approach of the present invention confers distinguishable benefits as compared with these other approaches in that the present approach provides, at least, significant increases in the energy molecule, ATP, which the targeted neurons can leverage to restart and/or in their normal processes. That is, one of the many benefits of the present approach, in addition to selecting laser parameters suitable for the patient and applying those parameters in scheduled, targeted laser applications, is to administer a therapy regimen whereby energy molecules are delivered only to affected neurons, where the energy molecules are delivered such that they cause a long lasting effect in patient improvement (as evidenced by the data in the present application). By properly scheduling the applications, the neurons can use the introduced ATP over a lengthy time period. Magnetic pulsation and electrical stimulation, on the other hand, work by disrupting the normal neuronal processes, shocking the neuron, if you will, and merely resetting it (in the cases of magnetic treatments), or in the case of electrical stimulation, delivering electrical currents (not unlike electroconvulsive therapy, or ECT) to various parts of the brain, with indiscriminate effects on neurons. Further, there is limited data, if any, indicating that the numerous patient conditions discussed in this application can show improvement using either magnetic pulsing or electrical stimulation.

Neuropsychiatric disorders treated by methods described herein include but are not limited to: Dementias (Alzheimer's, Lewy Body, Vascular, Frontotemporal), Aphasia, Parkinson's Disease, Cerebellar dysfunction, Agnosias and Apraxias, Mood disorders, Attention Deficit Disorder, Autism, Anxiety Disorders (including but not restricted to Obsessive Compulsive Disorder and Post Traumatic Stress Disorder), Traumatic Brain Injury, Chronic Traumatic Encephalopathy (CTE), disorders of wakefulness, Thalamo-cortical disconnection syndrome, Epilepsy, Stroke, and Multiple Sclerosis.

Because in theory there may be risk to the patient of applying such therapy indiscriminately (risk due to both lack of efficacy and the consequent lack of medical recovery, and also due to inappropriate use in inappropriate areas of the brain, with negative clinical consequences), the qEEG is used to narrow the location and parameters of application.

After a round of such treatments, a follow-up qEEG is applied to the patient and is used to determine success of the initial round and whether additional issues have arisen or improved. Based on the level of success, additional round(s) of laser treatment may be applied (iteratively as described here so as to continue success for the patient).

The present invention is comprised of a laser system, where different lasers of differing wavelengths may be available for application. The laser system may include electronics directed by a processor for focusing laser light based on selected parameters. The processor may be within the laser system or outside the system and in communication with the laser system.

The processor of the present invention may actually be a plurality of processors providing processing capability.

In the present invention, based on inputs such as but not limited to diagnoses, symptoms, MRI results, and qEEG results, the processor of the present invention selects a series of parameters for the laser application as well as selecting the quantity, duration, and frequency of laser application. In one embodiment of the present invention, the system of the present invention includes a database which encompasses a relationship between combinations of diagnoses, symptoms, MRI results, and qEEG results; patient types; conditions; and laser parameters such as but not limited to frequency, wavelength, power, and type of pulsing; as well as quantity, duration, and frequency of laser application, including wattage and joules. Based on database lookups and processing such data by the processor, a candidate set of parameters and application may be provided by the processor to the laser system and/or to a practitioner.

MRI is used at least in part to make sure the laser is safe (there are no venous anomalies etc. that would make laser unsafe), and the qEEG or equivalent provides refinement and can be used to more narrowly identify those areas, tracts, or networks for the laser application.

The database of the present invention may physically be a combination of databases and as new patients are provided with treatment, new symptoms identified, and so on, the database can be updated, either automatically or manually. The database itself is preferably a relational database but may take alternatively structured forms as well.

Further, the processor of the present invention can obtain data from test results, including post laser treatment results to identify patient changes, such as changes in brain tracts, which can be used to refine selection by the processor from the database. Further, machine learning can be used to refine processor functioning.

Also, although the processor can initially identify proposed parameters and application, a practitioner might change the approach, such as due to logistical reasons, and this change can be logged in the patient record in the database for later use.

In addition, the system for testing a patient (e.g., the qEEG) can be a part of the overall system of the present invention as well.

The present invention includes a sequence of steps, as described below. The steps described should be viewed as exemplary as there could be variation from patient to patient based on the patient's conditions, age, gender, and many other factors, and the steps themselves may occur in different sequences.

At a high level, the steps of the process following initial examination fundamentally include (but are not limited to) determining a target tissue and approach for treatment as follows:
a) Perform an MRI of the brain and obtain and analyze results to be sure no vascular abnormalities, masses, or other abnormalities are present on the surface or deeper in the brain.
b) Identify cortical areas of over activity and under activity and correlate with specific symptoms
c) Identify DTI tracts (Diffuse Tensor Imaging) which are over/under active and correlate with specific symptoms
d) Determine a sequence of which areas to treat first; e.g., treat over active areas first. In the preferred approach we compare the symptoms of the patient to both the function of Brodmann Areas (BA) with abnormal function, as well as the abnormal neuronal tracts on the DTI. We then identify the name and function of each abnormal tract. If the symptoms correlate with the function of the tract or the BA, then that area is considered a potential target for laser application. Areas of the greatest disturbance, symptomatically and neuronally may be targeted last.
e) Determine which areas are primary (based on symptoms, degree of abnormality, location, tract/cortical function, the history of the development of the symptoms, etc.) and which are secondary for treatment.
f) Determine what pulse frequency (Hz) to use; what wavelength (810 nM to 1064 nM) in which area; how many joules to deliver (range of 1-60 CM²); what wattage (5 W-30 W); what frequency of treatment (2/day to 1 per week). Pulse frequency may be determined by the frequency of the target tissue (determined at least in part by qEEG or equivalent results), by the pathology, and whether one wants to suppress or activate the target. For example, if alpha frequency is excessive in a particular location we preferably pulse at 10 Hz and apply the treatment more frequently. 40 Hz will reduce delta and theta activity, while elevating alpha, beta and gamma.
   If theta or delta frequency is excessive in a particular location we preferably use 40 Hz.
   If alpha, beta, or gamma frequencies are deficient we preferably use 15-40 Hz.
   Frequency and number of joules used in treatment are preferably determined based on the patient response. If the patient is over activated, the number of joules is reduced. Wattage is preferably determined by the highest amount tolerated by the patient without heating of the skin.
   The number of joules is initially set at zero level in order to determine an adverse placebo response. Absent an adverse placebo response, we preferably begin with a number of Joules per CM² (e.g., 3 J/CM²), and over the course of several sessions, taking into account the patients reported response, increase the J/CM² to the target of 60 J/CM².
   The wattage is preferably selected at the highest level tolerated by the patient without skin warming.
   Frequency of treatment is preferably determined by patient response and whether suppression of neuronal function is part of the plan (in which case we use more frequent treatment).
   Following 1-10 treatments, a repeat qEEG is performed to determine tissue response.
g) Tissue response is determined by the frequency in which the cortical or DTI tract disturbance appears, i.e., if the superior longitudinal tract DTI is underactive in the alpha band, we could elect to treat with 10 HZ light. If the person has Parkinson's disease we could elect to treat with 40 Hz in the specific locations identified. If the person has Alzheimer's we could elect to treat with 40 Hz or 10 Hz. If we want to reduce delta and theta, or elevate alpha, beta, gamma, we may use gamma frequency (40 Hz). Selection of alpha, beta, gamma, delta, and/or theta is based on the qEEG results, diagnosis, and clinical and qEEG response.
h) Assess patient response to prior treatment before each treatment

The process may begin with identifying the patient's symptoms, and the history of the development of the symptoms. With that information in hand, a qEEG is performed which, together with the symptoms, becomes useful in identifying the patient's dysfunctional tracts/BAs, including start and end points on the skull and level of activity (at least in a relative sense) of the tract. If a tract is overactive, one could apply an inhibitory light. The inhibitory quality of light is determined by pulse frequency and frequency of application. A 40 Hz light inhibits delta and theta. A 10 Hz light can be inhibitory in high doses of frequency or activating in low doses or frequency. If the tract is underactive, one preferably would use a stimulating frequency (e.g., a 40 Hz light will increase activity of beta and gamma neuronal activity), while concurrently taking into account other conditions of the patient. In some cases continuous wave (no pulse) may be used. In addition, the qEEG identifies the frequencies of under or over activity, so a laser is applied correspondingly. The frequency applied depends on where and what type of disturbance appears in the qEEG.

In the process of the present invention one would identify (automatedly or otherwise) the targeted area, choose the wavelength of the light (e.g. 810 nM) and apply a level of energy, preferably 6-60 joules/CM². It is important to recognize that only a small percentage of the applied energy will actually be administered to the patient, and it is preferable to start at a low level to determine tolerance of the patient. A follow up examination with the patient, typically in about 2 days, is used to determine the patient's tolerance to the applied light. Assuming the patient tolerated the treatment successfully; the number of joules/CM² may be increased.

In the preferred approach to the present invention, the light is applied a plurality of times, with potentially adjusted parameters depending upon patient tolerance. After application, typically and preferably but not limited to 1-10 treatments, a subsequent qEEG is administered to the patient. This second qEEG may show full normalization of the targeted areas (in which case treatment may be terminated) or partial normalization (in which case additional treatments would be given and a 3^{rd} qEEG would be used to determine the end of treatment). A second qEEG, along with a change in symptoms, could also show that the treatment needs to be modified or moved to a new area of the brain, following the criteria described above. In cases with underlying pathological processes, such as Alzheimer's Disease, where the brain is under a continuous and chronic assault due to genetic and other factors, chronic maintenance laser treatments with intermittent qEEGs may be used.

The present approach is usable for a variety of ailments, including Alzheimer's, PTSD, prosopagnosia, Parkinson's disease, stroke, traumatic brain injury, depression, and the ones described in the examples herein among others. Of note, the following examples are merely exemplary and the present approach could be applied to other maladies and case studies related to over or under activity of neuronal circuits, or optimization of normal circuits. Improvement is measured via objective measures such as improvement from baseline on computerized cognitive testing (e.g., CNS Vital Signs), neurometric testing (e.g. Cambridge Face Recognition test, Boston Naming Test, etc.), as well as normalization of the qEEG. Improvement is also measured by subjective reports of resolution of symptoms and improved function. Examples of improvement include:
A. JL (see case below) who presented with depression and memory problems (early cognitive decline due to cardiovascular disease). Following his Fx-Hylane program we asked him to self-assess any improvement and his response was “My memory is my new super-power.” Thinking that he might be exaggerating, we were able to have him repeat the CNS Vital signs test (see below). His memory had improved over the course of the one year's treatment:

| CNS Vital Signs | May 21, 2019 | May 12, 2020 |
|---|---|---|
| Computer Test | Percentile Score | Percentile Score |
| Composite Memory | 55 | 82 |
| Verbal Memory | 66 | 95 |
| Visual Memory | 45 | 53 |

B. AK (see case below) presented with a lifelong social phobia, and what appeared to be a paranoid schizoaffective disorder. The qEEG identified abnormalities in the coherence of neuronal white matter tracts which process facial recognition, the emotional valence of a face, and reading, even after he was on the FX aspect of the program. He was given 4 laser treatments (see below) and he reported a gradual dissolution of the facial distortions beginning at the 4^{th} treatment. Within one month all facial distortions were gone and he was able to interact with people with significantly greater ease, and a subjective reduction in his social phobia and paranoia, which has enabled him to be more social. In an unsolicited comment, he reported a marked improvement in his reading speed, which is a reflection of the improved function of the targeted neuronal tract.
C. PM (see case below) presented with mild cognitive impairment, including difficulty with memory, a 7 year history of acquired prosopagnosia (difficulty recognizing faces), and absence seizures. Following the application of the Fx portion of the program, she was treated with laser; The laser was targeted to specific areas in the frontal lobe and right temporal area which required her to shave her head. Within minutes after the first treatment she reported her acquired prosopagnosia cleared. This indicates that the neurons involved were in a living but marginal state, and provision of ATP via the targeted laser, provided enough energy for them to resume function. With a total of 25 treatments, her memory returned to normal (demonstrated by normalization of the hippocampal function on qEEG, see figure), her absence seizures were cleared without medication, and the prosopagnosia was eliminated so that she reported being able to follow along in movies (previously very difficult, since she could not recognize faces). Her post treatment Cambridge Facial Recognition score was 52 with an accuracy rate of 72%, with an average for young adults being 80% accuracy (Score of 58) placing her in the normal or low normal range at the end of treatment. She continues to run her business, which is expanding.
D. JS (see case below) is a 71 year old male with Parkinson's Disease; At the time of the evaluation his gait was irregular, he experienced spatial disorientation, cognitive decline, essential tremor, REM sleep behavior disorder, and nocturnal myoclonus. After laser treatment #3, he reported “My ataxia (symptoms of degenerative disease) may have slightly improved.” After 4 laser treatments he reported “I was having trouble buttoning the shirt buttons, but I notice that is largely (70%) better. I still have some trouble, but not very much.” After 5 laser treatments he reported “Cognitively, I am fine.” After laser #6 he reported “My left hand is moving a bit more in the last week, and cognitively I am much improved. My tremor has improved by 5-10%.” After the 7^{th} laser treatment, he reported “I also noticed that my cough headache (he would get a headache with each cough) which I have had for 8 years, appears to have gone.” The patient was treated at specific locations (C3-C4- and Cz) gradually increasing from 500 Joules to 6,000 Joules at 40 Hz (based on the symptoms and local abnormalities in both cortical and DTI connectivity). We have pre-post video footage demonstrating improvement. This patient had a total of 8 laser treatments;
E. PB presented as a 72 year old woman with advanced Lewy Body Dementia, with a Mini-mental status exam (MMSE) score of 22/30, frequent confusion, inability to finish sentences, irritability and physical aggression, inability to do simple things. After the first laser treatment the family reported: “when the phone rings the caller ID shows up on our television. Before the laser treatment she could not read the ID on the TV. However after the laser treatment she is able to read the ID. I think it is an improvement in her ability to pay attention to detail after two days she was unable to read or find the ID on the TV. “PB” was more responsive to conversation, more aware of things around her, had more energy and was occasionally finishing her thoughts. On the third day after the treatment she had some deterioration.” This indicates that the duration of the benefit was under 2 days, requiring increased frequency of treatment. Due to limitations of the husband's employment laser treatment was not initiated again until more than 3 months later. After this treatment the husband again and reported that “after the Laser “PB” was more attentive, had more energy, and more awareness. She was more talkative on the way home and was finishing sentences. This lasted for about four days and then there was a gradual drop off.” Once again there was a hiatus for approximately three months due to the husband's employment situation. At that time the patient was having a great deal of anger, sadness, hitting, and wandering out of the house. She was demonstrating poor hygiene. She was having difficulty swallowing pills and it was difficult to wake her in the mornings. The husband reported that “after the laser treatment she knew where the car was. This was very unusual. She did the dishes spontaneously on her room which she hasn't done in months. She put them away and dried them and 50% of them were in the right place. She had more energy and was able to stay up until 9 PM last night. She woke me to go to bed which is very unusual. She hasn't known who I am but yesterday called me by my name twice. She has been able to unhook her pants clasp and go to the bathroom which she has not been able to do. She got in the car this morning and expressed that it was a long drive. She has been answering questions with a yes or no and somehow her expression seemed different. Her anger is definitely reduced and she has generally had more energy and the stronger voice. She is saying her prayers and overall has increased energy and alertness.” After the second laser treatment the husband reported that she had a great deal of energy after the last treatment. They went home and the power was out. She asked him who he called and he said “Comcast”. After an hour and a half of no power she said “this is ridiculous”. It was very appropriate. In the evening he told her “I love you”, and she laughed and said ‘she loved me too’. This was the first time in a long time.” The patient's daughter noticed the energy in her voice that day. The patient arose early at 7 AM which is about 1½ hours earlier than her recent behavior. She was tired most of the day and very subdued but did not show any anger. Two days after the laser treatment she noticed that her nails were dirty. She had had no interest in self-care. She let me dress brush her hair and pull hairs from her chin. She then went on to pull hairs from her chin. This morning she woke up and knew who I was. She fixed her hair today. She noticed that she has gray in her hair and she is clearly more aware of her grooming. There is more spontaneous language and when she goes to bed at night she is not angry any longer. She actually brushed her teeth on her own.” After the third laser treatment the same changes were reported, and additionally the husband reported that she is exhibiting some spontaneous speech. He reported that on the way to the office she stated “‘Look how high the corn is”. She's talking more and asking more questions. She is smiling more and the evening before the office visit she allowed me (the husband) to put on her pajamas. She continues to pay more attention to her appearance.” After the fourth laser treatment her husband reported continued improvement with attention to detail (“she noticed my zipper was down on my pants and she notices we didn't have the dog leash when we were coming to your office. She noticed she did not have her seatbelt on and she knew she was coming to see. She's more interested in getting out and she's remembering her dog's name. She is more talkative. There are some full spontaneous sentences.”) Following the next laser treatment the husband reported that “Saturday was one of the best days we have had. She didn't wake up angry. When she did get angry I asked her to stop and she did. We were laughing. She let me hold her. She let me change her underwear but would not let me change her pants. She is brushing her teeth without with prompting. She was able to catch an error in her speech and she seems more aware. During the good days she understands a lot.” Following two more laser treatments the husband reported that she continues to talk more and her awareness and alertness continue. He stated “yesterday was a great day for us”. Overall things are improving and she has had no anger aside from one episode. “I have not been hit.” The patient herself stated “I am doing very great”. At this point the laser treatment was again interrupted due to the patient's husband's employment situation. The patient and her husband returned for additional laser treatment six weeks later and the husband reported before the resumption of this laser sequence that “the overall trend is downward”. Following the first laser in the series, the patient reported two days later “she did some reading yesterday but no writing. Yesterday was a great day and there was no anger. She was more alert and observant going home after the treatment. There was some kicking.” There was a continual pattern of good days after the laser treatment and a reduction in aggression. After a few more laser treatments the patient's husband stated “The violent anger has subsided 75 to 80% except for the day we left the office. For the last several days there has been no hitting or kicking. For the last four days there's been no arguing and she's letting me do things and she's pretty calm. It's been a good several days.” The laser treatment was again interrupted due to the husband's employment for 2.5 months. She had three additional treatments which she responded to in a similar manner as previous treatments. But once again the treatment was interrupted due to the husband's employment situation. The patient returned to treatment several months later, after the husband retired, but her behavior was quite unruly and aggressive; it was not possible to treat her in an office setting without causing great disruption to other patients.
F. JV presented as a 72 year old woman with a neurological diagnosis of mixed dementia with multiple etiologies including a) Alzheimer's disease (APOE3/4); b) fronto-temporal dementia (inappropriate friendliness, aphasia, confabulation, and prominent frontal-temporal lobe volume on MRI, along with global atrophy) and c) vascular dementia (multiple T2 subcortical and periventricular lesions suggestive of microvascular disease). She showed significant memory impairment (MOCA score 14/30) which began 11 to 12 years earlier, but had accelerated 3 months prior to the initial visit. The patient was not aware that she was having a problem. The patient had a series of 26 laser treatments, along with the FX program, hyperbaric oxygen treatment (HBOT) and brain games (Brain HQ twice daily). The patient's daughter reported better social behavior with more appropriateness but no improvement in her short term memory. Patient's husband reported that he did not see improvement and in fact felt there was some deterioration. The patient's daughter continued to report improving social appropriateness. Repeat qEEG after 10 laser treatments showed improvements including better frontoparietal coherence, improved cerebellar function. Phase coherence had not improved but hippocampal function, which was the site of most dysregulation, at 20 Hz showed a 50% improvement in connectivity. Additionally there seemed to be more differentiation between the left and right and a functional reintegration of the hemispheres. The thalamo-cortical regulation appeared to be within normal limits and the excessive frontal power was normalized. Despite these changes the patient's husband did not notice any improvement in memory. After 13 laser treatments, 3 months into the Fx aspect of the program, it became clear that the patient's husband was not controlling the patient's diet appropriately. Her husband was instructed again in dietary improvements and within a few days the husband reported there was some general overall improvement with the patient carrying on with full sentences and being more active than she was. “She is more cooperative.” The daughter reported “my mother's conversation is becoming more layered with more complexity and more engagement. This is a big difference from a year ago. For example she does not repeatedly say the same thing to our grandson on the drive to and from your office. Her conversation shows differentiation, a little movement, a subtle thing. I see overall a general well-being improvement.” Problems of noncompliance continued and the patient refused to use her CPAP machine for her sleep apnea. In addition it became clear that the high levels of mold in the home were never addressed by the husband. The patient had high levels of mold toxins including ochratoxin A which is neurotoxic, in her urine. These mycotoxins matched the types of mold found in the home environment. The husband was very sluggish in getting the problem remediated and as of the date of this report has still not remediated the problem. Dietary indiscretion is continued and patient's husband after 19 lasers reported more confusion and memory problems. A third qEEG after laser treatment #20 (while diet and mold issues were still not addressed properly) revealed that the brain was improved in most regards including the fronto-temporal disconnection. However in the alpha and low beta range the memory and attention networks were the same or even worse (in the areas that were improving on qEEG #2). Because the laser treatment has been improving in the patient, as seen in the qEEG and the clinical picture, it became clear that external factors such as diet and neurotoxic mold were likely playing a role. The patient continued to receive seven more laser treatments and was moved to her daughter's home (presumed to be mold free) where her diet could be under better control. After laser treatment #20 the husband reported “there is marked improvement! She can remember the date!” The daughter reported that she had not seen her in two weeks “there is less confusion and more clarity”. After laser treatment number 21 the husband reported “she's better today. Today she remembered what we had for dinner last night and her memory is 10 to 15% better.” After laser number 22 the patient's husband reported “she was out with our daughter Friday and she remembered what she did all day long. Four months ago she could not remember. We see improvement.” Following laser number 23 the patient's husband remarked that she makes progress and then backslides.” The patient remembered to bring in pictures for one of our staff members which she had promised a couple of days earlier. She put them aside and then was able to remember to bring them in, indicating a better functioning working memory.” A 4^{th} qEEG revealed significant improvement from the first qEEG. At this point the surface of the brain was normalized, as was the cerebellum. All frequency bands were improved by 20 to 50% with the exception of the beta-1 (low beta) band. The attention/default mode/executive function/face object recognition networks in the 12-14 Hz range were not improved. Laser treatment was interrupted due to office closure as a result of COVID-19. Repeat testing of mold toxins revealed that the mold toxin from her own home had cleared from her body following the move to her daughter's home. This correlated with her improvement when she moved to her daughter's home. This demonstrates the necessity of using the Fx (Functional Medicine) aspect of the program, in coordination with the Hylane treatments. Within 3 weeks following termination of the laser treatments, the family began to report a deterioration in the patient's memory. Four weeks after termination of laser treatment, the HBOT was stopped. The patient's decline accelerated. TGF-Beta 1 (a mark of inflammation) was markedly elevated; the family reported disorientation and confusion with a drastic change. The patient's vitamin D level was low indicating noncompliance with her supplement regimen, or malabsorption. Additionally, the family learned that the patient had been hiding snacks in the basement since moving to the daughter's home.
G. KM presented as a 62 year old female PhD, concerned regarding new onset head tremor, anxiety, trouble multi-tasking, low levels of energy, inability to sustain attention and concern regarding cognitive decline. She complained of frequent upper respiratory tract infections (URIs). Her treatment included normalization of thyroid function, analysis of genetics which indicated a genetically based glucocorticoid resistance. Via use of glucocorticoids and thyroid hormone her energy normalized and her frequent URI's were eliminated. Use of thiamine injections eliminated the head tremor. After 40 HBOT treatments her qEEG showed improved frontal connectivity, and normalized fronto-parietal connectivity. The qEEG signature of earlier brain injury was absent; After 26 laser treatments the patient reported “my brain is working better I feel more positive. I am able to stave off negative thoughts without so much effort. I am less apt to forget little things. I feel more together and am able to take risks. I'm less afraid and I'm seeking challenges.” However, her 4^{th} qEEG demonstrated that elevated theta (associated with decreased attention, difficulty with sustained attention) continued. The laser treatment was halted, HBOT was continued and the patient initiated neuronal exercises, in this case, neurofeedback. After 20 sessions there was normalization of theta activity, and marked improvement in sustained attention. The patient reported being able to sit down for several hours and read a book for the first time in her life. At the time of this writing she had read three books over the course of three weeks. Because there was no underlying pathophysiological process promoting neurodegeneration, there is no need for continued HBOT, laser or neurofeedback.
H. RH: The patient presented as a 67 year old female with a family history of fronto-temporal dementia; He complained of subjective cognitive decline, with no objective evidence supporting that as well as difficulty with spatial orientation. He described this problem as a tendency to be somewhat unaware of his location in space “in a subtle way”. When he would take a walk with his wife, or be in the grocery store with his wife, she would keep a safe distance so he would not bump into her, or step on her heels. He had a history of traumatic brain injury in the right temporal area, and this was evident on his qEEG. The qEEG showed a hypo-coherence at 10 HZ in the right temporal area. The skilled motor movement network at 10 HZ was normalized, as shown on the progressive changes between qEEGs 1-3, following 14 laser treatments to FP-1/FP2 and T4 using 10 Hz, and 40 Hz indicating recovery from the traumatic brain injury.
I. AC is a 62 year old woman who presented with complaints of fibromyalgia, fatigue, anxiety, confusion, and poor memory. She had a concussion at age 6 (dove into a pool and hit head, ill for 2 days) and at age 25 (car accident) which precipitated migraines (which cleared after 13 years), along with neck and shoulder pain. She was treated with the Fx arm of the program with modest relief. She was treated with laser treatments. After 4 laser treatments she reported an alleviation of pain by 30%, a mild reduction in anxiety (5-10%), better exercise recovery. After her 6^{th} laser treatment she reported increased ability to retain what she reads. After the 7^{th} laser treatment the patient reported “My confusion is gone.” A repeat qEEG indicated a need to reduce the laser frequency due to hyper-polarization, and decreased coherence in Alpha (8 Hz) in the right orbito-frontal cortex. This was consistent with new onset inappropriate laughter after laser #5. Frequency of treatment was reduced from 3 per week to 2 per week. The patient had a total of 8 laser treatments, and was the treatment was terminated due to office closure due to COVID-19. The patient maintained the reduction in pain (35% improvement), absence of confusion, and slight improvement of anxiety one month after termination of laser treatment. Inappropriate laughter was eliminated as soon as laser treatment frequency was reduced.

In some embodiments, a neuropsychiatric disorder treated by the method(s) of the present invention(s) includes at least one of: stroke, mechanical or emotional trauma, nutritional deficits, toxins, traumatic brain injury, hormonal imbalances, inflammation, aging, and genetics.

In some embodiments, the methods further include recording brain activity prior to, during, and/or after each treatment. In some embodiments, recording brain activity is preferably carried out by an equivalent to quantitative electroencephalography (qEEG).

In some embodiments, administering functional medicine may further be included in the method, and potentially includes administering pharmaceutical, nutraceutical, and/or physical procedures to treat abnormal digestion, nutrient deficiency, immune system dysfunction, toxins, mitochondrial dysfunction, hormonal dysfunction, genetic vulnerabilities, epigenetic/methylation deficits, structural problems, or any combination thereof.

In some embodiments, the methods of the present invention utilize a laser having a collimated beam. Preferably, the laser has a wavelength of about 780 nm to about 1100 nm. In some embodiments, the laser has a wavelength of about 810 nm.

In some embodiments, the methods of the present invention use a laser having a wattage of about 5 W to about 30 W. In some embodiments, the methods of the present invention preferably utilize a laser having a wattage of about 25 W. In some embodiments, the methods of the present invention utilize a laser having a wattage that increases from about 5 W to about 40 W over the duration of treatment. In some embodiments, the increase in power is gradual. For example, if a patient is treated with a laser with a wattage of 5 W, delivering a specific number of joules to a specific location, at a specific pulse frequency due the specific tissue requirements, and this is tolerated well, the subsequent treatment could utilize an amendment of the parameters, such that, for example, the wattage is increased to 10 W; Thus, depending on the patients response and the tissue response, this could be increased in the next step to 15 W, or reduced back to 5 W, or some intermediary value.

Another example of tissue driven changes would be changing from continuous wave to pulsed frequency. At 10-15 W, using a continuous wave (no pulse) about 0.45%-2.9% of the 810 nm light penetrates 3 cm of tissue (Henderson, Neuropsychiatric Disease and Treatment, 2015:11, 2191-2208). Should the tissue require less energy , pulsing at 10 Hz reduces the dose of light delivered at 3 cm from 2.9% to 2.4%. Choosing a different wavelength (e.g.,1064 nM) to achieve greater depth of penetration is an alternative option should this be indicated by objective parameters discussed above.

In some embodiments, the methods of the present invention utilize a laser administered in a pulse wave form. In some embodiments, the methods of the present invention utilize a laser operating in a Delta wave at about 1 Hz to about 4 Hz. In some embodiments, the methods of the present invention utilize a laser operating in a Theta wave at about 5 Hz to about 9 Hz. In some embodiments, the methods of the present invention utilize a laser operating in an Alpha wave at about 10 Hz to about 12 Hz. In some embodiments, the methods of the present invention utilize a laser operating in a Beta wave at about 13 Hz to about 30 Hz. In some embodiments, the methods of the present invention utilize a laser operating in a Gamma wave at about 40 Hz.

In some embodiments, the claimed method(s) encompass treating a neuropsychiatric disorder in a subject that includes any or all of administering a baseline cognitive test, administering hyperbaric oxygen treatment; administering laser treatment, administering cognitive exercises, and repeating the cognitive test, at least after said administration.

In some embodiments, the claimed method(s) encompass treating a neuropsychiatric disorder in a subject that includes simultaneously administering a laser to a subject's brain and recording brain activity. In some embodiments, the claimed method(s) further include administering functional medicine to a subject, analyzing brain activity of a subject, determining a treatment location for a laser application; and/or selecting laser parameters (e.g., wavelength, waveform, power, and/or duration of exposure). In some embodiment, the method(s) of the present invention include using a laser having a wavelength of about 810 nm.

In some embodiments, the method(s) include administering a qEEG before and after one or a series of laser treatments. In some embodiments, the method(s) of the present invention include adjusting the location(s) and/or parameters of the laser (or other modalities) based on the tissue-qEEG analysis.

In some embodiments, the present invention is a method of correcting physiological and other systems additionally through functional medicine (Fx) (e.g, providing nutrients and hormones; reducing destructive inflammation via assessment and treatment of the gastrointestinal tract, infection(s) and various sources of inflammation; identifying and eliminating toxins, addressing epigenetic function and methylation, structural problems, genetic vulnerabilities) which is then followed application of the HBOT (“Hy”), laser administration (“La”) and neuro-cognitive exercises (“ne”) (collectively, “Fx-HyLane”) program (e.g. an HBOT session to increase oxygen and blood flow, activates stem cells, etc.). In some embodiments, the claimed methods comprise performing a qEEG, and based on the areas of the brain requiring attention to achieve a desired outcome, a laser treatment is applied at a specified location(s) and with a specific parameter(s) (determined by analysis of qEEG, a subject's medical history, diagnosis, and/or performance objectives), to increase ATP production (and activate all the other mechanisms) at the specified areas. In some embodiments, a neural system is stimulated with a specific exercise(s) designed to challenge a desired neural pathway(s) to use the increase in ATP and oxygen production provided (and all the other physiologic benefits of the Fx-Hylane system) to create neuronal enhancement of function, at a specified location(s), network(s), and/or pathway(s). Methods encompassed by the embodiments of the present invention can provide building blocks with functional medicine, can provide precise targeted laser treatment, and can further direct neural change at specified locations of a subject's brain.

In some embodiments, subsequent to treating a subject with functional medicine, a subject's brain activity is recorded and analyzed. In some embodiments, a 19 channel qEEG is performed using software, such as but not limited to Neuroguide™ software, and an amplifier, such as but not limited to a Deymed amplifier, to record a subject's brain activity. In some embodiments, data is recorded under conditions where the subject's eyes are open for about 5-60 minutes and closed for about 5-60 minutes. The time range disclosed is not limiting and only reflects empirical data. A person of ordinary skill in the art would understand that the time required to obtain sufficient artifact free data depends on the nature of the subject and ultimately rests on the total amount of artifact free data that can be ascertained during a recording session. Functional medicine for the purpose of the present invention includes but is not limited to methods of treating physiological symptoms that include abnormal digestion, nutrient deficiency, immune system dysfunction, toxins, mitochondrial dysfunction, hormonal dysfunction, genetic defects, epigenetic/methylation deficits, structural problems (e.g., sleep apnea, fatty acid imbalances affecting the cellular membrane), or a combination thereof. Functional medicine can further include administering pharmaceutical or nutraceutical compounds or treatment by physical procedures. For example, some embodiments include administering nimodipine to treat symptoms of traumatic brain injury. Other embodiments include, for example, hyperbaric oxygen therapy (HBOT) to diffuse axonal injury.

Analysis of the qEEG using NeuroGuide™ (Applied Neuroscience, Inc.) software (or equivalent) can provide insight to the nature of a patient's disease and areas of normal brain function and can be used, along with desired behavioral outcomes, to determine the parameters of the laser use. Parameters of laser use that can be varied include but are not limited to wavelength, wattage, waveform (continuous wave, or pulse), pulse frequency (if a pulse wave is applied), the total energy applied (e.g., in Joules), area of application (e.g., square centimeters), location(s) on the head, duration of treatment (including both the duration of a single treatment or total treatment time), and/or frequency of treatment (time between treatments).

In some embodiments, the laser is preferably a collimated beam having one or more wavelengths in the range of about 780 nm to about 1100 nm. In a preferred embodiment, the laser has one or more wavelengths in the range of about 800 nm to about 850 nm. In a most preferred embodiment, the laser includes a wavelength of about 810 nm.

In some embodiments, the claimed method(s) include using a laser having a power of about 1 W to about 40 W. In some embodiments, the claimed method(s) include using a laser having a power of about 10 W to about 30 W. In some embodiments, the claimed method(s) include using a laser having a power of about 25 W. In some embodiments, the power of the laser can increase during the treatment, either during a treatment or during a course of treatments (e.g., treatment can begin using a laser at a lower wattage and increase to a higher wattage). In some embodiments one or more of the laser parameters can be altered based on qEEG, or the subjects reported response.

In some embodiments using a pulse wave form, the method(s) comprise using a laser having a Delta pulse frequency of about 1 Hz to about 4 Hz, a Theta pulse frequency of about 5 Hz to about 9 Hz, an Alpha pulse frequency of about 10 Hz to about 12 Hz, a Beta pulse frequency of about 13 Hz to about 30 Hz, and/or a Gamma pulse frequency of about 40 Hz.

The targeted surface location for laser application (on a subject's head) can be determined by analyzing a qEEG and/or by consideration of a subject's past medical history and diagnosis. For example, analysis of a qEEG can reveal areas of abnormal brain function in a subject. In some embodiments, the primary locations causing abnormal brain function can be determined based on a qEEG and a subject's past symptoms and diagnosis. For example, in some embodiments, a qEEG, medical history, and diagnosis can confirm a patient's reduction in neuronal coherence, and/or can indicate an area(s) or tract(s) of a patient's brain to receive laser administration. In some embodiments, a qEEG, a patient's medical history, and/or diagnosis can lead to a determination that specific areas of a patient's frontal lobes should be targeted to normalize that patient's parietal lobes. In some embodiments, the claimed methods include administering certain frequencies of laser radiation in a certain location(s) on a patient in order to inhibit and/or stimulate specific brain activity in a subject. In some embodiments, a patient's cerebellar area can be selected for irradiation using a laser having low-power (e.g., about 5 W to about 25 W) and lower total energy (in Joules) than would otherwise be administered. In some embodiments, the claimed methods include optimizing specific performance goals and/or brain function, and the networks and/or brain structures serving a specific function(s) can be treated and/or targeted using Fx-Hylane, including the laser. In some embodiments, the methods include practicing psychomotor exercises before, concurrent with, and/or after laser treatment so that the energy (ATP) provided by the laser can be channeled into the neuronal circuitry which is the focus attention. In some embodiments, an area(s) of interest (e.g., aberrant Broadman Areas (BA), or aberrant networks, or neuronal tracts, specific lobes, specific performance networks) can be translated to the international 10/20 system, and a cap can be placed on a subject to identify and demarcate a specific location(s) for administering the laser. In some embodiments, the present invention includes determining the etiological location(s) of a disorder or desired performance enhancement, and not areas of abnormal activity that reflect areas which may only reflect where the brain is compensating.

In the present invention, the method of administration of laser is determined by analysis of serial qEEG data at baseline and through the course of treatment. The qEEG data allows us to determine the specific locations where laser should be applied, what pulse frequency should be used, what wave length is most effective, and what frequency of treatment is most effective, and when to alter, pause or terminate treatment.

In some embodiments of the current invention, methods of treatment include methods of treating impulse control disorders and aggression attributed to Lewy Body disorders using qEEG analysis and laser treatment. In some embodiments, methods of treatment improve other disorders, such as those with Attention Deficit Disorder with hyperactivity. Without wishing to be bound by theory, it is also likely that some embodiments can treat specific populations or occupations. For example, a soldier or police officer (e.g., engaging with a terrorist situation or hostage situation, see the AK case as an example) faced with multiple potential opponents/targets needs sufficient response inhibition to discern which targets are indeed threats and which are not, before taking action. By optimizing the function of specific circuits (e.g., the salience network, the emotional-limbic fear network, enhancing frontal lobe function), the soldier or officer would have a reduction in limbic reactivity, more efficient function of the salience network, allowing for reduced cognitive load and higher order information processing, with the result of fewer errors and unwanted casualties.

In some embodiments, a qEEG is repeated after about 1-20 laser sessions or more and the protocol for treatment can be modified based on the result(s) observed by the qEEG.

In some embodiments, an area of application can range from about 3 cm² to about 60 cm². In some embodiments, an area of application can range from about 30 cm² to about 40 cm². In some embodiments, an area of application is about 35 cm².

In some embodiments, the present invention includes a method of treating a neuropsychiatric disorder in a subject that includes administering a baseline cognitive test to the subject, administering hyperbaric oxygen treatment, administering laser treatment, and/or administering cognitive exercises, neurofeedback, and optionally repeating the cognitive testing.

Cognitive testing may include but is not limited to, if the laser is administered, for example, to specific areas of the frontal lobes, testing functions such as planning, organizing, and working memory. For example, objective computer based testing may be used, such as CNS Vital Signs, or objective psychological testing can be included. In some embodiments, the claimed methods include administering laser treatment to a specific area(s) of the temporal lobe, and the subject undertaking exercises, for example, concerning language function including generating complex sentences, explaining paragraphs, and/or recalling a memory. Such exercises or testing may be performed prior to and/or after treatment, or both, and/or may be conducted during the course of treatment using the claimed methods. In instances in which the laser is administered to specific areas of the cerebellum, for example, cognitive testing can include a subject imagining a task and describing the task while the subject's eyes are closed, or performing physical tasks that can assess a subject's reaction time.

In some embodiments, the claimed methods include treating a neuropsychiatric disorder in a subject comprising simultaneously administering a laser to the subject's brain and recording brain activity before, during, and/or after the treatment. Recording brain activity while administering laser treatment provides real-time analysis and adjustment of the treatment. In some embodiments a subject would wear a portable device for capturing such activity for analysis. While not wishing to be bound by theory, there can be an immediate and clear amplitude change (increase in energy and activated neurons) in a region of the brain exposed to laser irradiation. Further, in some embodiments irradiation of a subject's brain according to the claimed methods can induce clear amplitude changes in a second region of a subject's brain that is not exposed to irradiation. Analysis of these effects can provide further insight to specific treatment parameters, and is an aspect of the qEEG-guided laser methodology of the invention.

In some embodiments, the method of administering laser treatment and simultaneous recording of brain activity further includes the use of single electrodes to record brain activity. In some embodiments, multiple electrodes or a “Combi-Cap” can be used to record brain activity. The Combi-Cap allows the application of laser during recording of the qEEG without the interference of cap materials. In some embodiments these electrode devices or corresponding devices could be used to allow for real time measurements of the laser application and its effect. Such measurements can be made by one or more of such devices and could be relayed to a processor for calculating and determining the impact of such laser applications. This allows real time measurement of the effect of the laser. In other embodiments, such measurements could alternatively or additionally be taken after laser treatments, such as periodically.

In some embodiments, the method of treating a neuropsychiatric disorder in a subject by simultaneously administering laser to the subject's brain and recording brain activity further includes steps of functional treatment of the subject, analyzing brain activity of the subject, determining a treatment location, and/or selecting the laser parameters.

### FURTHER EXAMPLES

These detailed descriptions serve to exemplify the above. These detailed descriptions are presented for illustrative purposes only and are not intended as a restriction on the scope of the invention.

### Example J

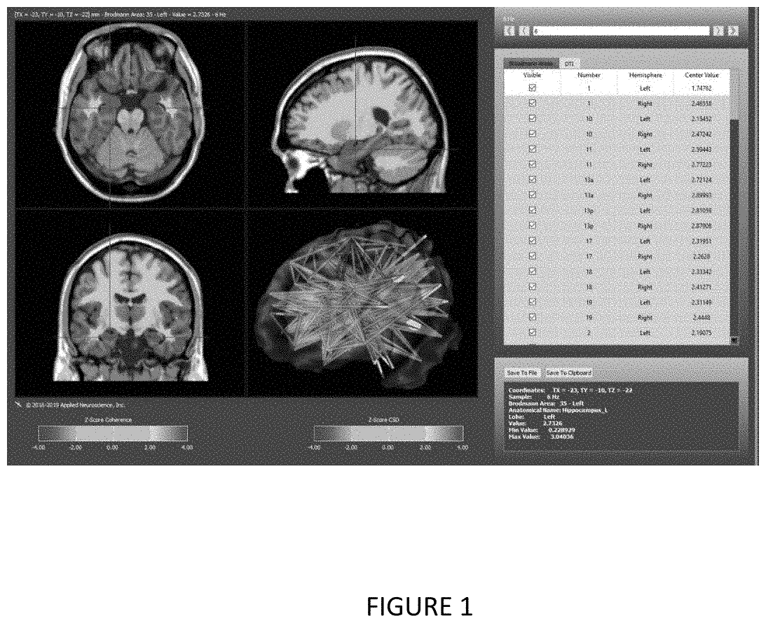

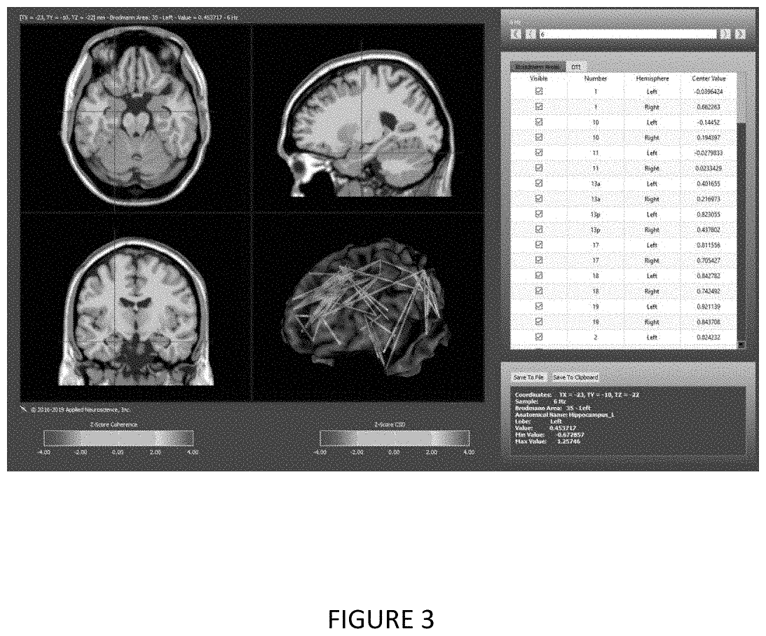

A patient who had a variety of problems (absence seizures, a traumatic brain injury, a 7 year history of prosopagnosia [trouble recognizing faces], mild cognitive impairment and strong vulnerability to Alzheimer's based on APOE4 genetics and family history) was treated with functional medicine (Fx), which included normalization of nutrient deficiencies (a very high need for lipoic acid, B2, B6, Folic Acid, B12, Manganese, Tyrosine, Arginine, Carnitine, DGLA, and a moderate need for Vitamins, A, C, E, B1, B3, Magnesium; treatment of iron overload; correction of zinc:copper:ceruloplasm in imbalance; correction of gastrointestinal inflammation; treatment of hormonal deficiencies (hypothyroidism, low DHEA/testosterone/melatonin); treatment of undermethylation; treatment of sleep apnea; treatment of mitochondrial deficiency). Subsequently, a 19 channel qEEG, using Neuroguide software and a Deymed amplifier, was used to record brain activity data of the patient with the patient's eyes open for 10 minutes and with the patient's eyes closed for 10 minutes. As shown in FIGS. 1 and 3 (left)—THIS SECTION COULD BE REDUNDANT AS IT DESCRIBES THE CASE OF PM STARTING AT THE BOTTOM OF PAGE 41, abnormal brain activity was concentrated at the frontal and temporal lobe regions. After analysis of the qEEG in consideration of the patient's diagnosis and symptoms, the area for laser treatment was determined to focus on specific areas of both the frontal and temporal region. Moreover, pulsing of an 810 nm laser to both the frontal and temporal area, at 10 Hz, which was the frequency of the neuronal populations at which there was deficient coherence and other parameters were abnormal, in the specific areas selected; and an initial setting of 10 Hz and wattage of 25 W was selected due to the need for deeper penetration and matching the neuronal frequencies needing attention (10 Hz), while maintaining higher energy delivery (25 W). The area of each target (determined by analysis of the qEEG and the patient's symptoms) was measured in cm, and a target of 60J/cm² was calculated. The specified areas (frontal and temporal lobes) were translated to positions on the patient's head consistent with the international 10-20 system, and a cap was placed on the patient to demarcate the areas, with a mascara pen, for administering the laser. The table below provides the treatment parameters for the first session using an 810 nm laser.

| | Surface | | | Fre- | | | |
|---|---|---|---|---|---|---|---|
| Area | Area | CW | | quency | Exposure | TOTAL | |
| Treat- | of | or | Laser | (if | time (ms | Joules | |
| ed | Laser | Pulse | Power | pulse) | on:off) | Admin. | Admin.r |
| FP1, | 40 cm² | P | 25 W | 10 Hz | 50:50 | 2400 J | Hedaya |
| FP2 | | | | | | | |
| F3, F4 | | | | | | | |
| (T3- | 36 cm² | P | 25 W | 10 Hz | 50:50 | 2160 J | Hedaya |
| F7) | | | | | | | |
| inf. | | | | | | | |
| (T5) | | | | | | | |

Two days later the initial treatment the patient reported a striking increase in memory retention, including facial recognition as well as recognition of physical features and surrounding environments.

The patient was interviewed before each laser session to determine clinical condition, and response to the prior treatment. Based on the response and qEEG analysis, the parameters of the laser and location(s) of administration were adjusted to address the needs of the tissue during each treatment session. After 25 treatments over the course of 2.5 months, normalization was exhibited in the patient's brain.

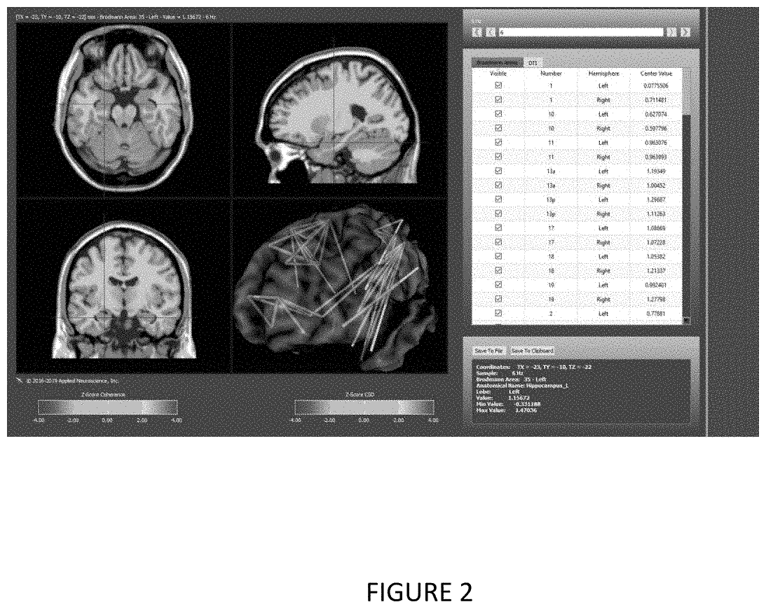

Patient interviews during the course of treatment demonstrated vast improvement in cognitive ability and treatment was discontinued. Surprisingly, three weeks after discontinuation of treatment, the patient maintained normalization cognitive ability and brain activity according to qEEG analysis and patient reported improvement. In particular, FIGS. 2 and 3 (right) demonstrated a distinct decrease in abnormal deviations of normal hippocampal values from 2.26 to 1.76 indicating improvements in memory dysfunction associated with Alzheimer's disease.

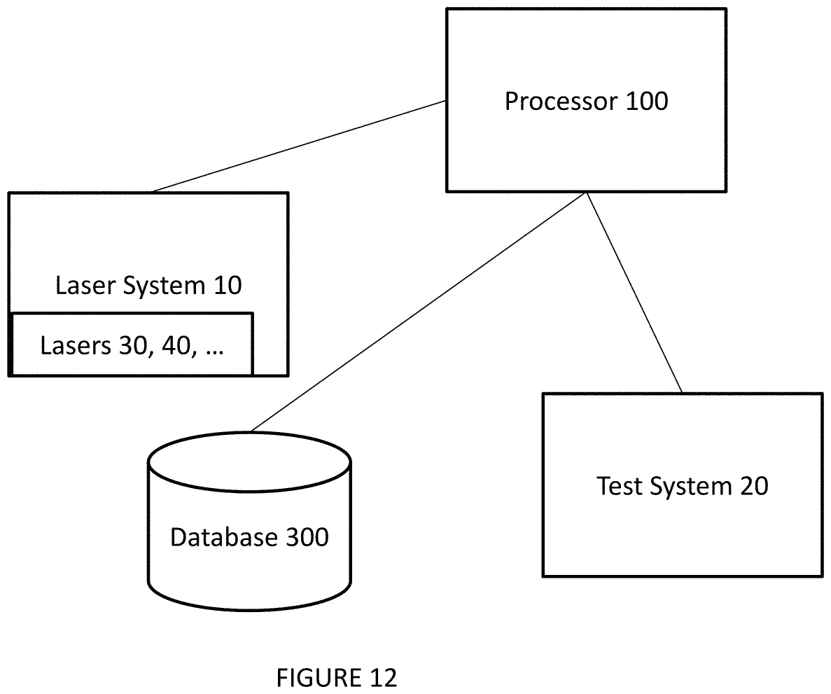

FIG. 12 provides a brief schematic of the present invention, showing processor 100 in communication with test system 20 and laser system 10 encompassing one or more lasers 30, 40, as well as database 300, where any of a plurality of lasers can be selected for use.

### Case: PM

A. Case History
1.1.1. Age & Diagnoses at initial presentation: Patient presented as a 58 year old divorced APOE 4 homozygote (2 genes for Alzheimer's Disease). She was a self-employed entrepreneur diagnosed with Mild Cognitive Impairment with a likely Alzheimer's (early stage) diagnosis, absence seizures and associative type acquired prosopagnosia (AAP) (7 year history of difficulty recognizing faces).
1.1.2. Medications
1.1.3. Presenting symptoms: Impaired memory, not recognizing people, forgetting major events, poor word recall, insomnia, difficulty with temperature regulation. Episodes of rage with loss of memory for the event, preceded by rising sensation in the abdomen, and unusual spiritual experiences (consistent with absence seizures).
1.1.4. Family History:
1.1.5. Physical Exam—
1.1.6. Cognitive testing: CNS Vital Signs—MOCA 27 (normal=30)
1.1.7. Imaging-MRI 2015: Lenticular cyst (in the putamen/globus pallidus) but otherwise normal; MRI w/o contrast Mar. 21, 2019 normal (no mention of lenticular cyst). Volumetric analysis (neuroreader) revealed left-right asymmetry of volumetric percentiles of temporal lobes (70.39 left vs. 60.33 right), amygdala (54.54 left vs. 62.33 right) and cerebellum (42.17 left vs. 48.47 right). Right and left hippocampi were symmetrical and normal in size, while total white matter was slightly below the mean at 46.71
1.1.1. Timeline:
   a. Premature birth (6 weeks)
   b. Age 4: head injury with loss of consciousness for several hours after paternal physical abuse
   c. Age 22—51: 3-4 glasses of wine per night
   d. Age 34—presentation: intermittent smoking
   e. Age 35: exposure to high levels of NO and CO2, and reduced levels of O2 for 6 months (Biosphere II)
   f. Age 46: depressive episode
   g. Age 49: menopause begins
   h. Age 51: acquired prosopagnosia begins
   i. Age 52: tinnitus
   j. Age 55: Sleep apnea (mild) diagnosis; worsening prosopagnosia
   k. Age 58: presents for treatment after severe episode of prosopagnosia
   l. Vasovagal seizures—age unknown

Lab Data
1.1.2. Functional Medicine Interventions:

Baseline qEEG (FIG. 1): The first qEEG was obtained after metabolic interventions were instituted for 6 months. Linked ears power spectral analyses deviated from the norm under both EC and EO conditions, with excessive power in bilateral frontal, temporal, parietal, and especially the right respective regions over a wide frequency range. The patient's Laplacian power spectral analyses deviated from the norm with excessive power from 6-9 Hz in bilateral frontal regions (especially in the midline frontal region), bilateral temporal regions (especially in the left temporal region) and bilateral occipital regions (especially in the right occipital region). The patient's EEG amplitude asymmetry, coherence and EEG phase deviated from the norm, especially in frontal, temporal, parietal and occipital relations. Elevated coherence (reduced functional differentiation) was present in frontal, parietal and occipital regions. Reduced coherence (reduced functional connectivity) was present in frontal, temporal, parietal, and occipital regions. LORETA 3-dimensional source analyses were consistent with the surface EEG and showed elevated current sources in the left superior transverse temporal gyrus & primary auditory cortex with a maximum at 3 Hz (Brodmann areas, BA 41). Elevated LORETA current sources were present in the left primary auditory area Broadmann Area (BA) 21 with a maximum at 5 Hz. BA 21 has been associated with recognition of known faces (Technologies TC. Cortical Functions. (2012). Elevated LORETA current sources were present in the right superior temporal gyrus & middle temporal gyrus with a maximum at 6 Hz (BA 38). BA 38 is among the earliest affected by AD, and at the start of temporal lobe seizures (Technologies TC. Cortical Functions. (2012). Elevated LORETA current sources were present in the left inferior frontal gyrus (BA 45) with a maximum at 7 Hz, associated with face encoding (Technologies TC. Cortical Functions. (2012), and the right prefrontal lobe with a maximum at 9 Hz (BA 10).
1.1.3. Treatment: Fx-HyLane

Step 1: FX: Treatment started May 21, 2019

Step 2: HyLane: Nov. 13, 2019-Jan. 23, 2020

An Aspen Pinnacle laser with maximum power of 60 watts (W), pulse frequency range 1-50 Hz, and a collimated beam was used. Following informed consent, the patient was instructed to shave down to the scalp (in specific areas) to allow maximal light penetration. Fluence, location, and pulse frequencies were varied based on clinical responses and an algorithmic interpretation of the qEEG data. After routine assessment, treatments were administered 3 times per week.

Laser Treatment Log for PM: BOLD indicates change in settings.

| | | | | | | If | If Pulse | TOTAL | |
|---|---|---|---|---|---|---|---|---|---|
| Date of | Area | CM- | # Joules | CW or | | pulse | ms on/ | Joules | |
| Treatment | Treated | sq | delivered | Pulse | Watts | # Hz | ms off | Delivered | Comments |
| Jan. 15, | FP1, | 40 | 2400 J | P | 25 W | 10 HZ | 50:50 | 2400 J | |
| 2019 | FP2 | CM2 | | | | | | | |
| | F3, F4 | | | | | | | | |
| | T3-F7, | 36 | 2160 | P | 25 W | 10 HZ | 50:50 | 2160 | Got car sick; |
| | T5 | cm2 | | | | | | | |
| Jan. 17, | FP1, | 40 | 2400 J | P | 25 W | 10 HZ | 50:50 | 2438 | Visual Memory |
| 2019 | FP2 | CM2 | | | | | | | better after |
| | F3, F4 | | | | | | | | 1^{st} tmt |
| | T3-F7, | 36 | 2160 | P | 25 W | 10 HZ | | 2163 | |
| | T5 | cm2 | | | | | | | |
| Jan. 18, | FP1, | 40 | 2400 J | P | 25 W | 10 HZ | 50:50 | 2438 | Visual Memory |
| 2019 | FP2 | CM2 | | | | | | | being |
| #3 | F3, F4 | | | | | | | | maintained- |
| | | | | | | | | | better wellbeing |
| | | | | | | | | | AFTER tmt- |
| | | | | | | | | | commented she |
| | | | | | | | | | could remember |
| | | | | | | | | | the next |
| | | | | | | | | | treatment dates |
| | | | | | | | | | [surprised] |
| | T3-F7, | 36 | 2160 | P | 25 W | 10 HZ | | 2163 | |
| | T5 | cm2 | | | | | | | |
| Jan. 21, | FP1, | 40 | 2450 J | P | 25 W | 10 HZ | 50:50 | 2438 | Visual Memory |
| 2019 #4 | FP2 | CM2 | | | | | | | being |
| | F3, F4 | | | | | | | | maintained- |
| | | | | | | | | | better wellbeing |
| | | | | | | | | | AFTER tmt- |
| | | | | | | | | | commented she |
| | | | | | | | | | could remember |
| | | | | | | | | | the next |
| | | | | | | | | | treatment dates |
| | | | | | | | | | [surprised] |
| | T3-F7, | 36 | 2433 J | P | 25 W | 10 HZ | | 2163 | |
| | T5 | cm2 | | | | | | | |
| Jan. 22, | FP1, | 40 | 2450 J | P | 25 W | 10 HZ | 50:50 | 2438 | Visual Memory |
| 2019 #5 | FP2 | CM2 | | | | | | | being |
| | F3, F4 | | | | | | | | maintained- |
| | | | | | | | | | better wellbeing |
| | | | | | | | | | AFTER tmt- |
| | | | | | | | | | commented she |
| | | | | | | | | | could remember |
| | | | | | | | | | the next |
| | | | | | | | | | treatment dates |
| | | | | | | | | | [surprised] |
| | T3-F7, | 36 | 2413 J | P | 25 W | 10 HZ | | 2163 | |
| | T5 | cm2 | | | | | | | |
| Jan. 25, | FP1, | 40 | 2463 J | P | 25 W | 10 HZ | 50:50 | 2463 | Speed (response |
| 2019 #6 | FP2 | CM2 | | | | | | | time went from |
| | F3, F4 | | | | | | | | 69 mph to 79 |
| | | | | | | | | | mph) increased |
| | | | | | | | | | in Lumosity |
| | | | | | | | | | over previous |
| | | | | | | | | | high (previous |
| | | | | | | | | | high was after |
| | | | | | | | | | first laser) |
| | T3-F7, | 36 | 2163 | P | 25 W | 10 HZ | | 2163 | No more word |
| | T5 | cm2 | | | | | | | retrieval |
| | | | | | | | | | problems |
| Jan. 28, | FP1, | 40 | 2500 J | P | 25 W | 10 HZ | | 2500 | Attempt to |
| 2019 #7 | FP2 | CM2 | | | | | | | increase |
| | F3, F4 | | | | | | | | hippocampal |
| | | | | | | | | | theta rhythm |
| | T3-F7, | 36 | 2100 J | P | 25 W | 1 HZ | | 2100 | |
| | T5 | cm2 | | | | | | | |
| Feb. 4, | FP1, | 40 | 2400 J | P | 25 W | 10 HZ | 50:50 | 2400 J | |
| 2019 #8 | FP2 | CM2 | | | | | | | |
| | F3, F4 | | | | | | | | |
| Go back | T3-F7, | 36 | 715 | P | 25 W | 10 HZ | | 2160 | |
| to original | T5 | cm2 | | | | | | | |
| Left | | | | | | | | | |
| Temporal | | | | | | | | | |
| Feb. 6, | FP1, | 40 | 2400 J | P | 25 W | 10 HZ | 50:50 | 2413 J | Continued |
| 2019 #9 | FP2 | CM2 | | | | | | | improvement |
| | F3, F4 | | | | | | | | over time in |
| | | | | | | | | | memory and |
| | | | | | | | | | facial |
| | | | | | | | | | recognition |
| | T3-F7, | 36 | 715 | P | 25 W | 10 HZ | 50:50 | 738 J | |
| | T5 | cm2 | | | | | | | |
| Feb. 8, | FP1, | 40 | 2400 J | P | 25 W | 10 HZ | 50:50 | 2413 J | Continued |
| 2019 #10 | FP2 | CM2 | | | | | | | improvement |
| | F3, F4 | | | | | | | | over time in |
| | | | | | | | | | memory and |
| | | | | | | | | | facial |
| | | | | | | | | | recognition |
| | T3-F7, | 36 | 715 | P | 25 W | 10 HZ | 50:50 | 738 J | |
| | T5 | cm2 | | | | | | | |
| QEEG | Hyper- | | | | | | | | |
| | coher- | | | | | | | | |
| | ence @ | | | | | | | | |
| | T4 | | | | | | | | |
| Feb. 13, | Right | 24 | 120 J | P | 5 W | 10 Hz | 50:50 | 120 J | |
| 2019 #11 | Brodman | CM2 | | | | | | | |
| | Area 10 | | | | | | | | |
| | FP1, | 40 | 2400 J | P | 25 W | 10 HZ | 50:50 | 2413 J | Continued |
| | FP2 | CM2 | | | | | | | improvement |
| | F3, F4 | | | | | | | | over time in |
| | | | | | | | | | memory and |
| | | | | | | | | | facial |
| | | | | | | | | | recognition |
| | T3-F7, | 36 | 725 | P | 25 W | 10 HZ | 50:50 | 738 J | |
| | T5 | cm2 | | | | | | | |
| Feb. 22, | FP1, | 40 | 2400 J | P | 25 W | 10 HZ | 50:50 | 2413 J | Continued |
| 2019 #12 | FP2 | CM2 | | | | | | | improvement |
| Repeat of | F3, F4 | | | | | | | | over time in |
| previous | | | | | | | | | memory and |
| treatment | | | | | | | | | facial |
| | | | | | | | | | recognition |
| | T3-F7, | 36 | 725 | P | 25 W | 10 HZ | 50:50 | 800 J | |
| | T5 | cm2 | | | | | | | |
| | Right | 24 | 120 J | P | 5 W | 10 Hz | 50:50 | 125 J | |
| | Brodman | CM² | | | | | | | |
| | Area 10 | | | | | | | | |
| Feb. 27, | FP1, | 40 | 2400 J | P | 25 W | 10 HZ | 50:50 | 2425 J | |
| 2019 #13 | FP2 | CM2 | | | | | | | |
| Repeat of | F3, F4 | | | | | | | | |
| previous | | | | | | | | | |
| treatment | | | | | | | | | |
| | T3-F7, | 36 | 2400 J | P | 25 W | 10 HZ | 50:50 | 2413 J | |
| | T5 | cm2 | NB 3+ | | | | | | |
| | | | times the | | | | | | |
| | | | # of | | | | | | |
| | | | Joules as | | | | | | |
| | | | previous | | | | | | |
| | Right | 24 | 250 J | P | 5 W | 10 Hz | 50:50 | 330 J | |
| | Brodman | CM² | | | | | | | |
| | Area 10 | | | | | | | | |
| Mar. 1, | FP1, | 40 | 2400 J | P | 25 W | 10 HZ | 50:50 | 2413 J | |
| 2019 #14 | FP2 | CM2 | | | | | | | |
| Repeat of | F3, F4 | | | | | | | | |
| previous | | | | | | | | | |
| treatment | | | | | | | | | |
| | T3-F7, | 36 | 2400 J | P | 25 W | 10 HZ | 50:50 | 2463 J | |
| | T5 | cm2 | | | | | | | |
| | Right | 24 | 330 J | P | 5 W | 10 Hz | 50:50 | 335 J | |
| | Brodman | CM² | | | | | | | |
| | Area 10 | | | | | | | | |
| Mar. 4, | FP1, | 40 | 2400 J | P | 25 W | 10 HZ | 50:50 | 2413 J | No change |
| 2019 #15 | FP2 | CM2 | | | | | | | No cognitive |
| | F3, F4 | | | | | | | | proplems |
| | T3-F7, | 36 | 2400 J | P | 25 W | 10 HZ | 50:50 | 2400 J | |
| | T5 | cm2 | | | | | | | |
| | Right | 24 | 330 J | P | 5 W | 10 Hz | 50:50 | 365 J | |
| | Brodman | CM² | | | | | | | |
| | Area 10 | | | | | | | | |
| Mar. 6, | FP1, | 40 | 2400 J | P | 25 W | 10 HZ | 50:50 | 2413 J | No change |
| 2019 #16 | FP2 | CM2 | | | | | | | No cognitive |
| | F3, F4 | | | | | | | | proplems |
| | T3-F7, | 36 | 2400 J | P | 25 W | 10 HZ | 50:50 | 2400 J | |
| | T5 | cm2 | | | | | | | |
| | Right | 24 | 650 J | P | 25 W | 10 Hz | 50:50 | 650 J | |
| | Brodman | CM² | | | | | | | |
| | Area 10 | | | | | | | | |
| Mar. 6, | FP1, | 40 | 2463 J | P | 25 W | 10 HZ | 50:50 | 2463 J | No change |
| 2019 #17 | FP2 | CM2 | | | | | | | No cognitive |
| | F3, F4 | | | | | | | | proplems |
| | T3-F7, | 36 | 2425 J | P | 25 W | 10 HZ | 50:50 | 2425 J | |
| | T5 | cm2 | | | | | | | |
| | Right | 24 | 700 J | P | 25 W | 10 Hz | 50:50 | 700 J | |
| | Brodman | CM² | | | | | | | |
| | Area 10 | | | | | | | | |
| Mar. 11, | FP1, | 40 | 2500 J | P | 25 W | 10 HZ | 50:50 | 2500 J | Saw movie |
| 2019 #18 | FP2 | CM2 | | | | | | | and could |
| | F3, F4 | | | | | | | | not recognize |
| | | | | | | | | | faces. A |
| | | | | | | | | | deterioration |
| | | | | | | | | | Could not sleep |
| | | | | | | | | | last night after a |
| | | | | | | | | | “tiff with |
| | | | | | | | | | daughter’ |
| | T3-F7, | 36 | 216 J | P | 25 W | 10 HZ | 50:50 | 216 J | |
| | T5 | cm2 | Lowered | | | | | | |
| | | | the dose | | | | | | |
| | | | back to | | | | | | |
| | | | original | | | | | | |
| | | | settings | | | | | | |
| | Right | 24 | 300 J | P | 25 W | 10 Hz | 50:50 | 700 J | |
| | Brodman | CM² | | | | | | | |
| | Area 10 | | | | | | | | |
| Mar. 15, | FP1, | 40 | 2450 J | P | 25 W | 10 HZ | 50:50 | 2450 J | No benefit from |
| 2019 #19 | FP2 | CM2 | | | | | | | last tmt |
| | F3, F4 | | | | | | | | |
| | T3-F7, | 36 | 1175 J | P | 25 W | 10 HZ | 50:50 | 1175 J | |
| | T5 | cm2 | | | | | | | |
| | Right | 24 | 325 J | P | 25 W | 10 Hz | 50:50 | 325 J | |
| | Brodman | CM² | | | | | | | |
| | Area 10 | | | | | | | | |
| Mar. 18, | FP1, | 40 | 2442 J | P | 25 W | 10 HZ | 50:50 | 2442 J | Better since |
| 2019 #20 | FP2 | CM2 | | | | | | | yesterday |
| | F3, F4 | | | | | | | | |
| | T3-F7, | 36 | 1175 J | P | 25 W | 10 HZ | 50:50 | 1175 J | |
| | T5 | cm2 | | | | | | | |
| | Right | 24 | 325 J | P | 25 W | 10 Hz | 50:50 | 325 J | |
| | Brodman] | CM² | | | | | | | |
| | Area 10 | | | | | | | | |
| Mar. 19, | | | | | | | | | |
| 2019 | | | | | | | | | |
| qEEG | | | | | | | | | |
| Mar. 20, | FP1, | 40 | 2442 J | P | 25 W | 10 HZ | 50:50 | 2442 J | Better since |
| 2019 #21 | FP2 | CM2 | | | | | | | yesterday |
| | F3, F4 | | | | | | | | |
| | T3-F7, | 36 | 263 J | P | 25 W | 10 HZ | 50:50 | 1175 J | |
| | T5 | cm2 | | | | | | | |
| | Right | 24 | 388 J | P | 25 W | 10 Hz | 50:50 | 325 J | |
| | Brodman | CM² | | | | | | | |
| | Area 10 | | | | | | | | |
| Mar. 22, | FP1, | 40 | 2442 J | P | 25 W | 10 HZ | 50:50 | 2442 J | |
| 2019 #22 | FP2 | CM2 | | | | | | | |
| | F3, F4 | | | | | | | | |
| | T3-F7, | 36 | 263 J | P | 25 W | 10 HZ | 50:50 | 1175 J | |
| | T5 | cm2 | | | | | | | |
| | Right | 24 | 338 J | P | 25 W | 10 Hz | 50:50 | 325 J | |
| | Brodman | CM² | | | | | | | |
| | Area 10 | | | | | | | | |
| Mar. 25, | FP1, | 40 | 3013 J | P | 25 W | 10 Hz | 50:50 | 3013 | |
| 2019 # 23 | FP2 | CM2 | | | | | | | |
| | F3, F4 | | | | | | | | |
| | T3-F7, | 36 | 513 J | P | 25 W | 10 HZ | 50:50 | 513 J | |
| | T5 | CM2 | | | | | | | |
| | Right | 24 | 363 J | P | 25 W | 10 Hz | 50:50 | 363 J | |
| | Brodman | | | | | | | | |
| | Area 10 | | | | | | | | |
| Mar. 27, | FP1, | 40 | 3125 J | P | 25 W | 10 Hz | 50:50 | 3013 | Seems more |
| 2019 # 24 | FP2 | CM2 | | | | | | | organized |
| | F3, F4 | | | | | | | | today |
| | T3-F7, | 36 | 525 J | P | 25 W | 10 HZ | 50:50 | 513 J | |
| | T5 | CM2 | | | | | | | |
| | Right | 24 | 413 J | P | 25 W | 10 Hz | 50:50 | 363 J | Reports doing |
| | Brodman | | | | | | | | well |
| | Area 10 | | | | | | | | Still remembers |
| | | | | | | | | | the faces from |
| | | | | | | | | | the movie she |
| | | | | | | | | | watched. |
| Apr. 1, | FP1, | | 3011 | P | 15 W | 10 HZ | 50:50 | 3011 J | |
| 2019 # 25 | FP2 | | | | @ 810 | | | | |
| | F3, F4 | | | | nm | | | | |
| | | | | | 15 W | 10 Hz | | | |
| | | | | | @ 980 | | | | |
| | | | | | nm | | | | |
| | T3-F7, | | 538 J | | 15 W | 10 HZ | 50:50 | 538 J | |
| | T5 | | | | @ 810 | | | | |
| | | | | | nm | | | | |
| | | | | | 15 W | 10 Hz | | | |
| | | | | | @ 980 | | | | |
| | | | | | nm | | | | |
| | Right | | 521 J | | 15 W | 10 HZ | 50:50 | 521 J | |
| | Brodman | | | | @ 810 | | | | |
| | Area 10 | | | | nm | | | | |
| | | | | | 15 W | 10 Hz | | | |
| | | | | | @ 980 | | | | |
| | | | | | nm | | | | |

1.1.4. Follow Up qEEG

FIG. 1: This figure shows the baseline (pre-laser, post HYLANE) qEEG on Jan. 19, 2019. The left panel of the figure which as 4 quadrants (before any laser) shows two aspects. The orange areas in quadrants 1-3 reflect the areas of the surface of the brain that are over active in theta (6 Hz), a common finding in dementia. Importantly the red cross hairs are centered on the hippocampus, a primary site of dysfunction in Alzheimer's Disease. The rectangle on the lower right of the figure gives the values for the current source density of the left hippocampus, which is clearly abnormal at 2.73 standard deviations from the norm. The 4^{th} quadrant of the left panel depicts hypo (blue) and hyper (yellow) coherence via tubular connections between different Brodmann areas (surface of the brain) @ 6 Hz. The right aspect of the figure (all pink) depicts Brodmann Areas which are abnormal in current source density at 6 Hz.

FIG. 2: This figure shows the eyes open condition at 6 Hz on Mar. 19, 2020, after 21 laser treatments. Note the complete normalization of the current source densities. Additionally, there is a remarkable normalization of both hyper and hypocoherence. Most striking is the normalization of the left hippocampal current source density, which 1.1 standard deviations from the mean (normal).

FIG. 3: This figure, from Apr. 22, 2019, in the eyes open condition @ 6 Hz, demonstrates continued improvement 21 days after the last laser treatment. This is demonstrated by the lower Center Values in the different Brodmann Areas listed on the right side of the figure (e.g., BA 1, 10, 11, etc.), as well as the lower value of the hippocampal (where the red cross hairs are) center value in the rectangle on the lower right side of the figure. The function of this deep structure normalized, as shown by the normalization of the Center Value over the course of treatment, going from markedly abnormal value of 2.73 (figure PM 1), to 1.56 (figure PM 2), to 0.45 (Figure PM 3).
1.1.5. Clinical Outcome:

Objective:
See qEEG figures above
MOCA test was 27/30 at initial evaluation, and normalized (30/30) after HYLANE treatment (before laser).

Subjective:

After the first HILT the patient reported via email: “I was car sick when I left. It went away. After I left a client's home in the afternoon, I realized ‘I can remember the client—I could visualize his face, body, height, and what his teeth looked like, lots of details . . . . I would be able to pick him out of a crowd. Shocking. It's been so long since I had facial recognition like that. Yesterday I did an audit, and I can remember the husband. Today I did an audit and I can remember what the person looks like. ”

After the 4^{th} treatment: “There is definitely better facial recognition. I watched a movie last night and I can remember the actress's face, the 2 moles on her face. It is a definite improvement.”

After the 11^{th} HILT treatment: “I feel like I have gotten my brain back. I see improvement in little things all the time. With the functional medicine treatment I have more vitality, motivation, and energy—mostly affecting my body. With the light therapy, it's changing my brain. I see it in my life.”

After the 13^{th} treatment : “My memory improvement is occurring at such a rate that I am regaining memory capacity I never realized I had lost. Now people call me 2-3 weeks after an order, and I can remember what we discussed 2-3 weeks ago, specific things I saw in their house.”

After the 23^{rd} treatment: “My brain has changed. I can remember names and faces. Am I perfect? No. I have gotten lazy—I expect not to remember them so I don't look at people.”

Four months after the last HILT treatment (Jul. 26, 2019) the patient reported: “I don't feel the facial recognition is a problem anymore.” She reported that her business had expanded allowing her to hire three employees. “I made more money this year, so far, than in all of 2017”. Despite these improvements, she felt there was some regression, and she felt that “the laser treatments were terminated prematurely.”
1.1.6. Methodology: The locations targeted were of two types: Brodmann areas and deep neuronal tracts based on DTI. Targets were determined by the confluence of areas which were aberrant on the qEEG, and whether the function of the abnormal tracts coincided with the clinical symptoms. Because hypercoherence and elevated current source densities were prevalent, 10 Hz was used. We have determined that using 10 Hz with frequent treatment applications causes an inhibition of neuronal firing. The repeat qEEG after treatment #10 indicated some hyper-coherence in the right temporal area, which we suspect is a remote effect of the treatment. This area was targeted with a lower power (5 W @ 10 Hz) to provide a more modest amount of energy. Treatment was terminated when the eyes open qEEG had normalized and this was consistent with alleviation of symptoms.

### Case 2: JL

A. Case History
1.1.8. Age & Diagnoses at initial presentation: 65 Year Old Entrepreneur
   Vascular Dementia with Mild Cognitive Impairment
      1.1.8.1.1.1. Recurrent Unipolar Depression—rule out bipolar II Currently depressed (mild to moderate)
   Cardiovascular Disease
   Fibromyalgia
   History of alcoholism and marijuana use in remission
1.1.9. Medications: olopatadine, methylphenidate 5 mg, Androgel, buprenorphine,
   atorvastatin, tadalafil, Lunesta, DHEA,
1.1.10. Presenting Symptoms:
   Trouble with names, finding words, spatial disorientation,
   Focus
   Depression with anhedonia intermittently
   Pain in muscles and joints
   Low efficiency at work (very distractable)
   Cervical stenosis, psoriasis
1.1.11. Family History: Maternal Dementia, Paternal Bipolar Disorder; PGF—Paranoid
   Schizophrenia; Brother alcoholic
1.1.12. Physical Exam—impaired balance, Positive Rhomberg, reduced DTR's, reduced
   capillary reperfusion, onychomycosis, thrush, cold hands/feet.
1.1.13. CNS Vital Signs—all scores average; reaction time and reasoning below average
   SAGE test 20/22
   Beck Depression Inventory=16
1.1.14. Imaging—MRI (age 59): 2014: white matter lesions
1.1.15. Timeline
   Enuresis to age 12
   Age 15 (1969)—Depression onset—black periods intermittently, interspersed with
   periods of high productivity without hypomania
   Long history of alcohol abuse
   1990—Major Depression
   1992—possible hypomania
   2000—severe depression; successful treatment with Prozac
   2004—stop marijuana/ETOH
   2008—anterior and posterior cervical fusion
   2008—onstet of hypertension
   2001-2010—successful treatment with bubroprion
   2010-2018—metabolic syndrome, rapidly progressive cardiovascular disease
   2018—Mild Cognitive impairment (trouble with names, spatial disorientation,
      word finding,
1.1.16. Lab Data
   Mild pancreatic insufficiency
   Ceruloplasmin 12.7 (16-31)
   Copper 69 (72-166)
   Dysbiosis with candidiasis, klebsiella, Enterobacter and Blastocystis
   Various nutritional Deficiencies
   Copper 69 (72-166); Ceruloplasmin 12.7 (L)
   Free Testosterone 6.0 (6.6-18.1)
   ACTH—49.5
   C4a 942.7 (<650)
   Total IGG 534 (700-1600)
   Genetics: NR3C1: 50% of alleles (14/28) are single nucleotide polymorphisms conferring potential glucocorticoid resistance, as well as increased risk to depression, PTSD and immune illness.
1.1.17. Functional Medicine Interventions: Replace nutrients, treat dysbiosis, support
   with pancreatic digestive enzymes, probiotics, dietary change, normalize testosterone; Institute high intensity interval training 5 days per week, with strength training. Hyperbaric Oxygen Therapy; Hydrocortisone (glucocorticoid).
1.1.18. Baseline qEEG
   DX: Mild Vascular Dementia
   High levels of theta (7-8 Hz) in F3/F7
   Slowing at 21 HZ
   T6 slowing in right temporal area
      Hypo-coherence in >15 Hz: psychomotor slowing, inefficient cognitive function
1.1.19. Treatment: Fx-HyLane
   Step 1: FX: Treatment Started May 21, 2019
   Correction of diet
   Treatment of GUT Dysbiosis
   Nutrition—correction of deficits
   High intensity interval training
   Adrenocortical support with hydrocortisone, DHEA, pregnenolone
   Step 2: HyLane: Nov. 13, 2019-Jan. 23, 2020
   Hyperbaric Oxygen Therapy (HBOT)—1.4 ATA—80 treatments
   Laser therapy—start date: Nov. 13, 2019 (see laser treatment log below)

Laser Treatment Log: Patient Name: JL

Bold highlights indicates a change in parameters based on clinical/qEEG data

| | | | | CW | | If | If Pulse | TOTAL | |
|---|---|---|---|---|---|---|---|---|---|
| Date of | Area | | # Joules | or | | pulse | ms on/ | Joules | |
| Treatment | Treated | CM-sq | delivered | Pulse | Watts | # Hz | ms off | Delivered | Comments |
| Nov. 13, | F3, F7 | 96 CM² | 2100 | P | 15 W | 10 Hz | 50:50 | 2100 | |
| 2019 | | | | | | | | | |
| Laser # 1 | | | | | | | | | |
| Nov. 15, | F3, F7 | 16 × | 2800 J | P | 20 W | 10 Hz | 50:50 | 2870 J | Ultimate |
| 2019 | | 4 == 64 | | | | | | | target is 4800 |
| Laser # 2 | | CM² | | | | | | | for F3, F7, F4 |
| | F4 | 4 × 4 = | 800 J | P | 20 W | 10 Hz | 50:50 | 810 J | |
| | | 16 cm² | | | | | | | |
| Nov. 18, | F3, F7 | 16 × | 2800 J | P | 20 W | 10 Hz | 50:50 | 2880 J | Ultimate |
| 2019 | | 4 == 64 | | | | | | | target is 4800 |
| Laser # 3 | | CM² | | | | | | | for F3, F7, F4 |
| | F4 | 4 × 4 = | 800 J | P | 20 W | 10 Hz | 50:50 | 810 J | Patient |
| | | 16 cm² | | | | | | | reported after |
| | | | | | | | | | laser # 2 that |
| | | | | | | | | | he felt lighter, |
| | | | | | | | | | even under a |
| | | | | | | | | | significant |
| | | | | | | | | | stress |
| Laser # 4 | | F3 | 3500 J | P | 20 W | 10 Hz | 50:50 | 3510 J | Reports |
| Nov. 21, | | F7 | | | | | | | (before this |
| 2019 | | | | | | | | | treatment) |
| | | | | | | | | | less procras- |
| | | | | | | | | | tination; |
| | | F4 | 1000 J | P | 20 W | 10 Hz | 50:50 | 1020 J | |
| | | C3 | 800 J | P | 20 W | 10 Hz | 50:50 | 800 J | |
| | | C4 | 800 J | P | 20 W | 10 Hz | 50:50 | 800 J | |
| | | T6-not | | | | | | — | |
| | | doing | | | | | | | |
| | | yet | | | | | | | |
| Laser # 5 | | F3 | 3500 J | P | 20 W | 10 Hz | 50:50 | 3500 J | |
| Nov. 25, | | F7 | | | | | | | |
| 2019 | | | | | | | | | |
| | | F4 | 1000 J | P | 20 W | 10 Hz | 50:50 | 1000 J | |
| | | C3 | 800 J | P | 20 W | 10 Hz | 50:50 | 800 J | |
| | | C4 | 800 J | P | 20 W | 10 Hz | 50:50 | 800 J | |
| | | T6-not | | | | | | | |
| | | doing | | | | | | | |
| | | yet | | | | | | | |
| Laser # 6 | | F3 | 3500 J | P | 20 W | 10 Hz | 50:50 | 3500 J | |
| 11/75/19 | | F7 | | | | | | | |
| | | F4 | 1000 J | P | 20 W | 10 Hz | 50:50 | 1000 J | |
| | | C3 | 800 J | P | 20 W | 10 Hz | 50:50 | 800 J | |
| | | C4 | 800 J | P | 20 W | 10 Hz | 50:50 | 800 J | |
| | | T6-not | | | | | | | |
| | | doing | | | | | | | |
| | | yet | | | | | | | |
| Laser # 7 | | F3 | 4500 J | P | 20 W | 10 Hz | 50:50 | 4540 J | |
| Dec. 2, | | F7 | | | | | | | |
| 2019 | | | | | | | | | |
| | | F4 | 1000 J | P | 20 W | 10 Hz | 50:50 | 1000 J | |
| | | C3 | 800 J | P | 20 W | 10 Hz | 50:50 | 800 J | |
| | | C4 | 800 J | P | 20 W | 10 Hz | 50:50 | 610 J | |
| Laser # 8 | | F3 | 3500 J | P | 20 W | 10 Hz | 50:50 | 3500 J | Felt worse |
| Dec. 5, | | F7 | | | | | | | after last |
| 2019 | | | | | | | | | session |
| | | F4 | 1000 J | P | 20 W | 10 Hz | 50:50 | 1000 J | |
| | | C3 | 800 J | P | 20 W | 10 Hz | 50:50 | 810 J | |
| | | C4 | 800 J | P | 20 W | 10 Hz | 50:50 | 800 J | |
| Laser # 9 | | F3 | 3500 J | P | 20 W | 10 Hz | 50:50 | | |
| Dec. 9, | | F7 | | | | | | | |
| 2019 | | | | | | | | | |
| | | F4 | 1000 J | P | 20 W | 10 Hz | 50:50 | | |
| | | C3 | 1000 J | P | 20 W | 10 Hz | 50:50 | | |
| | | C4 | 1000 J | P | 20 W | 10 Hz | 50:50 | | |
| First T6 | | T6 | 800 J | | | | | | |
| treatment | | | | | | | | | |
| Laser # 10 | | F3 | 3500 J | P | 20 W | 10 Hz | 50:50 | 3500 J | |
| Dec. 12, | | F7 | | | | | | | |
| 2019 | | | | | | | | | |
| | | F4 | 1000 J | P | 20 W | 10 Hz | 50:50 | 1000 J | |
| | | C3 | 1000 J | P | 20 W | 10 Hz | 50:50 | 1000 J | |
| | | C4 | 1000 J | P | 20 W | 10 Hz | 50:50 | 1000 J | |
| 2nd T6 | | T6 | 800 J | | | | | 810 J | |
| treatment | | | | | | | | | |
| qEEG # 2 | | | | | | | | | |
| Dec. 13, | | | | | | | | | |
| 2019 | | | | | | | | | |
| Dec. 16, | | Fz-Cz- | 2500 J | P | 20 W | 30 Hz | | 2500 | |
| 2019 | | Pz | | | | | | | |
| Laser # 11 | | F4-F3 | | | | | | | |
| | | C3-C4 | | | | | | | |
| Change | | FP2- | 2000 J | P | 20 W | 20 Hz | | 2000 | |
| | | Fp1 | | | | | | | |
| Dec. 19, | | Fz-Cz- | 2500 J | P | 20 W | 30 Hz | | 2500 | After prior |
| 2019 | | Pz | | | | | | | treatment: |
| Laser # 12 | | F4-F3 | | | | | | | Reports better |
| | | C3-C4 | | | | | | | mood; some |
| | | | | | | | | | increased |
| | | | | | | | | | irritability; |
| | | | | | | | | | energy better |
| | | | | | | | | | in a sustained |
| | | | | | | | | | way; |
| | | FP2- | 2000 J | P | 20 W | 20 Hz | | 2000 | |
| | | Fp1 | | | | | | | |
| Dec. 23, | | Fz-Cz- | 2500 J | P | 20 W | 30 Hz | | 2500 | |
| 2019 | | Pz | | | | | | | |
| Laser # 13 | | F4-F3 | | | | | | | |
| | | C3-C4 | | | | | | | |
| | | FP2- | 2000 J | P | 20 W | 20 Hz | | 2000 | |
| | | Fp1 | | | | | | | |
| 3 rdT6 | | T6 | 800 J | P | 10 W | 10 Hz | | 810 J | |
| treatment | | | | | | | | | |
| Dec. 26, | | FP-1 | 1500 J | P | 20 W | 10 Hz | | | |
| 2019 | | | | | | | | | |
| Laser # 14 | | | | | | | | | |
| | | FP 2 | 1500 J | P | 20 W | 20 Hz | | | |
| | | Fz-Cz- | 2500 J | P | 20 W | 30 Hz | | 2500 | |
| | | Pz | | | | | | | |
| | | F4-F3 | | | | | | | |
| | | C3-C4 | | | | | | | |
| | | C4-T6 | 800 J | P | 10 W | 20 Hz | | 810 J | |
| Dec. 30, | | FP-1 | 1500 J | P | 20 W | 10 Hz | | | |
| 2019 | | | | | | | | | |
| Laser # 15 | | | | | | | | | |
| | | FP 2 | 1500 J | | 20 W | 20 Hz | | | |
| | | Fz-Cz- | 2500 J | P | 20 W | 30 Hz | | | |
| | | Pz | | | | | | | |
| | | F4-F3 | | | | | | | |
| | | C3-C4 | | | | | | | |
| | | C4-T6 | 1500 J | P | 10 W | 20 Hz | | | |
| Jan. 3, | | FP-1 | 2000 J | P | 20 W | 10 Hz | | | |
| 2020 | | | | | | | | | |
| Laser # 16 | | | | | | | | | |
| | | FP 2 | 2500 J | | 20 W | 20 Hz | | | |
| | | Fz-Cz- | 2500 J | P | 20 W | 30 Hz | | | |
| | | Pz | | | | | | | |
| | | F4-F3 | | | | | | | |
| | | C3-C4 | | | | | | | |
| | | C4-T6 | 1500 J | P | 10 W | 20 Hz | | | |
| Jan. 14, | | FP-1 | 2000 J | P | 20 W | 10 Hz | | 2000 J | |
| 2020 | | | | | | | | | |
| Laser # 17 | | | | | | | | | |
| | | FP 2 | 2500 J | | 20 W | 20 Hz | | 2508 J | |
| | | Fz-Cz- | 2500 J | P | 20 W | 30 Hz | | 2505 J | |
| | | Pz | | | | | | | |
| | | F4-F3 | | | | | | | |
| | | C3-C4 | | | | | | | |
| | | C4-T6 | 1500 J | P | 10 W | 20 Hz | | 1500 J | |
| Jan. 16, | | FP-1 | 2000 J | P | | 10 Hz | | 2000 J | |
| 2020 | | | | | | | | | |
| Laser # 18 | | | | | | | | | |
| | | FP 2 | 3500 J | | 20 W | 20 Hz | | 2508 J | |
| | | Fz-Cz- | 2500 J | P | 20 W | 30 Hz | | 2505 J | |
| | | Pz | | | | | | | |
| | | F4-F3 | | | | | | | |
| | | C3-C4 | | | | | | | |
| | | C4-T6 | 1500 J | P | 10 W | 20 Hz | | 1500 J | |
| Jan. 20, | | FP-1 | 2000 J | P | 20 W | 10 Hz | | | I did feel a |
| 2020 | | | | | | | | | definite lift |
| Laser # 19 | | | | | | | | | the day after |
| | | | | | | | | | this. More up |
| | | | | | | | | | and positive |
| | | | | | | | | | than I have in |
| | | | | | | | | | a long time |
| | | | | | | | | | (a burst of |
| | | | | | | | | | happiness- |
| | | | | | | | | | first time in |
| | | | | | | | | | months) |
| | | FP 2 | 3500 J | | 20 W | 40 Hz | | | |
| | | Fz-Cz- | 2500 J | P | 20 W | 10 Hz | | | |
| | | Pz | | | | | | | |
| | | F4-F3 | | | | | | | |
| | | C3-C4 | | | | | | | |
| | | C4-T6 | 1500 J | P | 10 W | 10 Hz | | | |
| Jan. 23, | | FP-1 | 3500 J | P | 20 W | 10 Hz | | 3500 J | |
| 2020 | | | (increase) | | | | | | |
| Laser # 20 | | | | | | | | | |
| | | FP 2 | 3500 J | | 20 W | 40 Hz | | 3500 J | |
| | | Fz-Cz- | 2500 J | P | 20 W | 10 Hz | | 2505 J | |
| | | Pz | | | | | | | |
| | | F4-F3 | | | | | | | |
| | | C3-C4 | | | | | | | |
| | | C4-T6 | 1500 J | P | 10 W | 10 Hz | | 1505 J | |

1.1.20. Follow Up qEEG (See Pre-Post Images)
   Blinking which was prominent in the first qEEG—is gone in qEEG #2
      29 Hz all the hypoconnectivity is far less visible
      The brain is much healthier (70% where we want it to be) in front to back information flow.
   The default mode network in 17 Hz—this is now a normal brain for his age;
      Hyper-coherence in the corpus collosum—no longer present.
      Strong evidence of normalization (patient notices he less pulled to sadness as his
      default).
1.1.21. Clinical Outcome:
   Objective:
   See qEEG FIGS. 1-4 (I do not know how the figures were renamed, but it cannot be FIGS. 1-4 can you look at a previous iteration?)
   SAGE test—22/22 (was initially 20/22)—vascular dementia SAGE score does not normally improve with time.
   CNS Vital Signs:

| | May 21, 2019 | May 12, 2020 |
|---|---|---|
| Test | Percentile Score | Percentile Score |
| Composite Memory | 55 | 82 |
| Verbal Memory | 66 | 95 |
| Visual Memory | 45 | 53 |
| Psychomotor Speed | 55 | 61 |
| Reaction Time | 10 | 23 |
| Complex Attention | 68 | 73 |
| Cognitive Flexibility | 40 | 47 |
| Processing Speed | 63 | 58 |
| Executive Function | 37 | 47 |
| Reasoning | 21 | 27 |
| Simple Attention | 70 | 70 |
| Motor Speed | 50 | 63 |

Comparison of DTI on qEEG 1-3 (Again, I do Not Know Which Figures These Refer to):
   10 Hz-12 Hz—very significant improvement
   Right frontal pole—underpowered (expected)
   15 Hz—much better
   20-21 Hz—much improved
   25 Hz—significantly improved
      Subjective:
      (Feb. 13, 2020)—20 days post laser
         Mood: Feeling pretty good; re-engaged with work; seems better—feel sharper;
         Word finding—pretty good; Seems normal—2 months ago, I would not have said that
         Names—improved by 20%
         Spatial disorientation—have not had that problem
      (Apr. 22, 2020)—3 months post laser
         Affect bright. Feeling generally good—no depression; Mood is good; More alert in the AM; “My memory is my new super-power!—Great recall”
         Distractibility—less.
         Continues the HBOT 3 times per week.
1.1.22. qEEG Images baseline—see left side of FIGS. 1-4 (same problem)
1.1.23. qEEG Images at termination of treatment—see right side of FIGS. 1-4,
1.1.24. Methodology—(Discussed Above)
a) The qEEG at each time point was analyzed to determine which areas were hyper-coherent or hypo-coherent
b) The qEEG at each time point was analyzed to determine which surface areas had elevated or reduced current density
c) Brain areas and neuronal tracts which were thought to be primary (i.e., causal) were identified and segregated from areas of dysfunction thought to be compensatory or secondary.
d) The primary neuronal tracts were identified and correlated to their termination points in or around specific Brodmann areas.
e) Surface areas that were determined to be primary (abnormal current density) were correlated with Brodmann areas.
f) Identified Brodmann Areas were converted to the 10/20 system
g) The patient shaved their head in the identified 10/20 system target areas
h) The targeted areas (e.g., F3/F7) were marked before each treatment with removable marker pen
i) The volume of the targeted areas (in CM²) was measured.
j) The maximal joules that could be administered per treatment were calculated as 60 J/CM²
k) The patient was initially treated with 50% or less of the maximum Joules.
l) Pulsed light was used due to deeper penetration and lower tissue heating.
m) Wattage was determined based on skin color, estimated thickness of skull but was always started low (10-15 W)
n) Pulse frequency was determined by the abnormal frequency of the target tissue
o) Fans, sweeping motions with the laser hand-piece and interruptions of the laser were used as needed to maintain normal skin temperature.
p) Skin temperature was monitored by touch every 30 seconds
q) qEEG was repeated and based on the analysis, parameters were modified

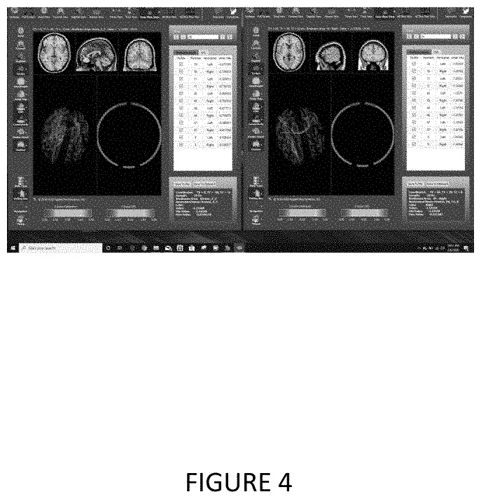

FIG. 4: At 24/25 Hz: Change between treatments #10 (Left) and #20 (right)—Many areas of DTI abnormality (blue) on patients right (left side of image) are gone.

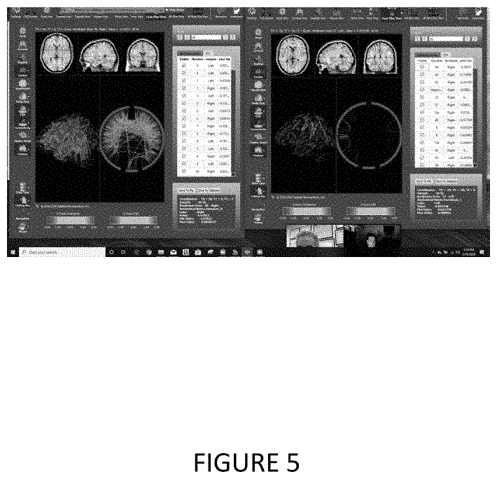

FIG. 5: Baseline (left) and after 20 laser treatments 26 Hz—areas of hypo-coherence nearly gone.

Explanation of FIGS. 4 and 5: Baseline (left) and after 20 laser treatments Salience Network Connectivity normalized. The function of the salience network is to select stimuli that are worthy of our attention. There are 2 images of the salience network: The top image at 24/25 Hz, and the bottom image is at 26 Hz. The two images show baseline (Dec. 13, 2019, on the left) before laser, and after laser treatments (Feb. 5, 2020, on the right) were completed.

FIG. 4: this depicts the DTI (diffuse tensor imaging) in the right and left hemispheres of the brain @ 24 and 25 Hz; this frequency is involved in higher order cognitive functions. The abnormality is consistent with malfunction of the patients salience network indicated by symptoms of his excessive attention to numerous stimiuli as if they are relevant both internally (e.g., his body, pain) and externally (fearful responses to external stimuli of minor importance, e.g., worry about innocuous comments a co-worker would make). Note in the left panel the high amount of DTI abnormalities indicated by the light blue and dark blue, with the right side of the brain being worse than the left side. Note in the panel on the right that the amount of DTI abnormalities are markedly reduced. This change correlated with the patient reporting moderate reduction in pain, and marked reduction worry about external events.

FIG. 5: The left panel (baseline) has two aspects. The left brain image, depicts hypocoherence (different than DTI in FIG. 4, as it reflects out of phase or dyssynchronous neuronal signaling, where DTI is a reflection of dysfunction in the actual white matter tracts themselves) via the light blue and dark blue tubular connections between different Brodmann areas (surface of the brain) involved in the salience network. The right aspect (of the left panel) depicts the same information in a connectome diagram in which the left hemi-circle shows the connectivity between various surface Brodmann Areas relevant to the salience network on the left side of the brain, and the right hemi-circle shows the connectivity between various surface Brodmann Areas relevant to the salience network on the right side of the brain. The right panel demonstrates almost complete normalization of the phase relationships in the salience network following the full course of qEEG guided laser treatments. This means that the surface areas of the brain involved in salience detection are now coordinating their function normally.

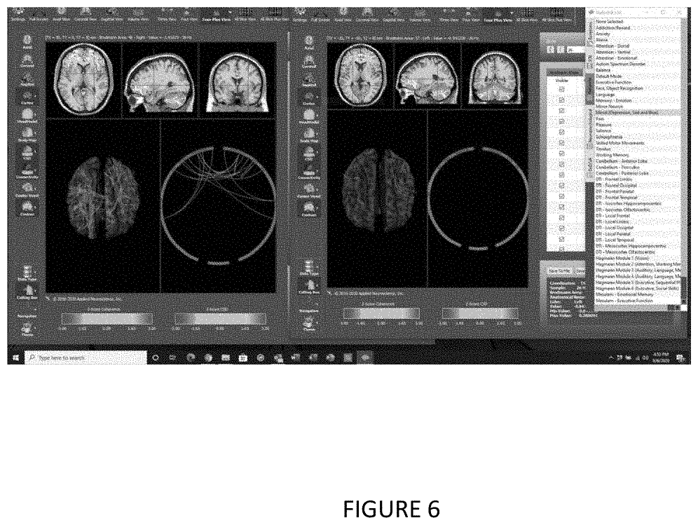

FIG. 6—This depicts the DTI (diffuse tensor imaging) in the right and left hemispheres of the brain @ 26 Hz in the mood/depression network; This frequency is involved in higher order cognitive functions. The abnormality is consistent with malfunction of the patient's mood regulation neuronal network indicated by symptoms of his having a chronically depressed mood. Note in the left panel the high amount of DTI abnormalities indicated by the light blue and dark blue, with the right side of the brain (shown on the left, since you are facing the patient) being more widespread than the left side of the brain, which is more confined, but more severe (dark blue). Note in the panel on the right (after laser treatments) that the DTI abnormalities are nearly eliminated. This change correlated with the patient reporting absence of depression.

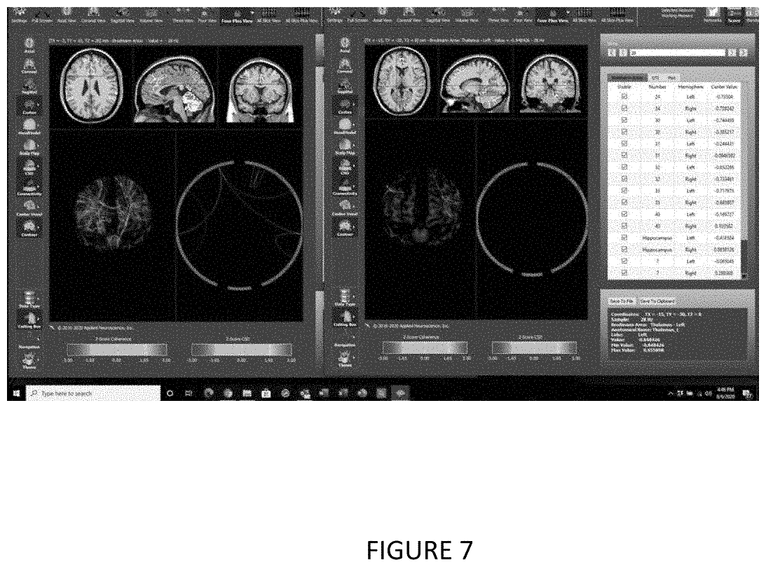

FIG. 7: This depicts the DTI (diffuse tensor imaging) in the right and left hemispheres of the brain @ 28 Hz in the working memory network; This frequency is involved in higher order cognitive functions, and working memory is important in the ability to remain on task, for executive function, and short term memory. The abnormality is consistent with malfunction of the patient's complaints of distractibility and poor efficiency at work, as well as complaints of short term memory problems. Note in the right portion of the left panel (the red circular area) the connection abnormalities indicated by the light blue and dark blue, with the right side of the brain a bit worse than the left side. Note in the panel on the right (after laser treatments) that the connection abnormalities are nearly eliminated. This change correlated with the patient reporting significantly improved memory, (consistent with his CNS vital signs test-re-test scores, and his text message to me [“my memory is my new super-power”]) and his increased efficiency at work.

### Case 5: AK

A. Clinical History
1.1.1. Age and Diagnoses at initial presentation: 21 year old college student
   Schizoaffective Disorder with Paranoia, and hypomania
   Social Phobia
   Recurrent Depression—in remission
   Obsessive Compulsive Disorder
   Grand Mal Seizures (Occipital) age 5, and febrile seizure X 1
   REM Sleep Behavior Disorder
   ADD
1.1.2 Medications: Luvox, Risperidone, Trazodone, Naltrexone
1.1.3. Presenting Symptoms: Social anxiety; isolation; nightmares; carbohydrate cravings; headaches; Perception of peoples facial expressions as menacing or demeaning.
1.1.4. Family History: Maternal: Eating disorder(M), depression (MGM, MGGM) ETOH
   (Great Uncle); Paternal: Bipolar/OCD (Uncle), depression; Depression/vascular
   dementia (GM), ETOH (3).
1.1.5. Physical Exam—within normal limits
1.1.6. CNS Vital Signs; Most scores average; visual memory low average; complex
   attention low average.
   BDI—11; YBOC=14; ADD=21
1.1.7. Imaging:
   MRI—2017 (age 20)—normal;
   SPECT Scan (age 15): Increased activity in anterior cingulate gyrus, and lateral pre-frontal cortices; Increased activity in thalami, basal ganglia, and temporal lobes (bilateral); Reduced inferior orbital PFC bilaterally;
   Decreased cerebellar activity.
1.1.8. Timeline:
   Early childhood: Socially anxious; Seizures alleviated with elimination of gluten; night terrors
   Age 3—Febrile Seizure
   Age 4—Mild head injury
   Age 3-5: Night terrors/tantrums
   Age 5—Grand Mal seizure
      Age 16: irritability, isolation
   Age 18—paranoid
      Age 19—leaves school; Antipsychotics/Lamictal—no help
   Age 20—wilderness program—paranoid delusions, Aud. Hallucinations; psychiatric hospitalization X 2
1.1.9. Lab Data
   Gastro-intestinal: Blastocystis; Pancreatic insufficiency; Elevated Beta-glucoronidase;
   Immune: VEGF <31 (31-86); +Anti-tubulin and +Anti-dopamine Ab; TGF-Beta 1 8400 (<2382)
   Nutritional: MCV=99, RBC 4.78 (4.14-5.8); Vitamin A—high; CU—0.74 (0.8-1.75)
   Sleep: Paroxysmal leg movements of sleep; reduced REM latency
   Methylation: Active folate low, SAH 50.8 (38-49); glutathione low; cysteine low;
   Endocrine Genetics: Multiple Variants in NR3C1, FKBP5, CRHr1/2, CRHBP, DIO2

| Genetic Finding | Meaning | To Do |
|---|---|---|
| NR3C1 (6/14 | Reduced genetic | Emotional Wellbeing |
| snp's) | responsiveness to | Therapy with Regina |
| | glucocorticoid (cortisol) and | Do enjoyable things |
| | consequent difficulty | Hydrocortisone |
| | handling stress, fighting | |
| | infection; Inc. risk of | |
| | depression | |
| SLC6A4 | Increased risk of | Consider reduction in |
| | depression; less benefit | Luvox |
| | from SSRI's | |
| SLC6A2 | Responsible for re-uptake | Heart Math |
| | of norepinephrine; | |
| | associated with adrenal | |
| | insufficiency and orthostatic | |
| | intolerance. | |
| TPH2 | Controls the first step in | 5-HTP |
| Tryptophan | making serotonin; | Bacopa Monnieri |
| hydroxylase 2 | Associated with OCD | Vitamin D |
| | | Increased protein to |
| | | Carb ratio |
| ESR1 | Associated in 4 studies with | Genistein (soy) |
| Estrogen | abnormal behavioral traits | |
| Receptor 1 | in males and females: | |
| | hypomania, delusions; A | |
| | definitive assessment of | |
| | mechanism cannot be | |
| | determined | |
| HNMT-Histamine | Reduced ability to degrade | Salacia oblonga |
| N-Methyltrans- | histamine in the central | SAMe |
| ferase | nervous system | B12 |
| | | 5-MTHF |
| FUT2 | FUT2 polymorphism leads | B12 |
| | ABH non-secretor status, | Probiotics |
| | increased susceptibility to | Bifidobacteria |
| | chronic diseases; gut | (e.g., align) |
| | microflora imbalance and | Avoid Alcohol |
| | less functional intestinal | Look into non-secretor |
| | membrane. Reduced B12 | diet (Peter D'Adamo— |
| | absorption, and increased | Eat Right for |
| | risk of H. Pylori | Your Type) |
| MAOA | Intolerance of | Before using |
| 3 SNP's | b12/SAMe/folic acid—even | B12/SAMe/Folates: |
| rs 3027399 | though needed—is likely. | Use high dose activated |
| rs 909525 | An initial improvement with | B2-riboflavin-5- |
| rs6323 | these agents may be | phosphate |
| MAOB | followed by a crash/brain | Magnesium |
| | fog | Glycine |
| | | Lithium Orotate |
| | | Ashwaganda |
| | | Avoid-curcumin |
| COMT (val)- | While Andrew has COMT | Magnesium |
| increased activity | polymorphisms they may | B3 |
| CACNA1B, | be compensated for by the | P5P |
| ESR1-reduced | other genes listed to the left | Avoid caffeine |
| COMT activity | | Avoid caloric restriction |
| GCH1 | | Avoid Green Tea |
| GAD1 | Glutamate Decarboxylase- | P5P |
| | necessary to make GABA | |
| | (calming) | |
| VDR (Vitamin D | | Magnesium |
| Receptor) | | B3 |
| Multipl snps | | P5P |
| Taql, Taq1, | | B2 |
| A1012G, Apal, | | Vit D |
| Bsmal 1024 | | Butyrate |
| CBS | Inability to process | P5P |
| | homocysteine into | SAMe |
| | cystathione (transulfuration) | |
| | SAH AND Insulin further | |
| | reduce this activity | |
| CFH-complement | Helps control activation of | AVOID selenium |
| factor H ARMS2 | the complement system- | |
| BHMT | Helps convert | Folic |
| | Homocysteine to | Zinc |
| | Methionine; Defect | Betaine |
| | contributes to increased | Phosphatidyl-Choline |
| | methionine | |
| PER2 (Period | Affects circadian rhythm | Melatonin |
| homolog 2) | | RE-check—was very |
| CLOCK | | high initially |

1.1.10 Fx-HyLane Interventions:
   Treat the dysbiosis,
   Normalize methylation (18 months), correct diet; Nutritional supplementations;
   Hydrocortisone, thyroid hormone, DHEA, pregnenolone;
   See chart above.
1.1.11 Baseline: qEEG #1:
   Impression: PRIMRARY PROBLEM: Front to back (occipital/parieto-frontal)
   dysfunction, affecting visual perception of faces (Brodmann area 17 and frontal
   44/45), resulting in secondary stress on the:
   a) corticothalamic integration areas (excess need for integration due to danger),
   b) fronto-pontine (danger),
   c) cortico-striatal (prepare for action)
      DTI Analysis of qEEG #1 Shaded areas are areas of treatment

| Frequency | Excess/Deficient | DTI Tract Name/Function | Intervention |
|---|---|---|---|
| Delta | Excess | Left Frontal | Thiamine |
| 1-4 | | Left Cortico-thalamic tract | (helps carb |
| STEP 2 | | Left Cortico-striatal tract | metabolism- |
| | | | thiamine helps |
| | | | striatum) |
| | | | Left Frontal |
| | | | 40 Hz (Gamma) |
| | | | FP1-F3-F7 |
| | | | Start with 250J |
| Theta | Excess | Right Parieto-pontine tract | 40 Hz Gamma |
| 8-9 | | | @ P4 |
| | | | Start with 250J |
| Alpha | Massive Diffuse | Bilateral but L > R | None |
| 10-11 Hz | Excess | Left frontal (Right is NORMAL) | |
| | | Left Corticothalamic tract | |
| | | Left Cortico-striatal tract | |
| | | Corpus Collosum | |
| Low Beta | Deficient | Right Superior Longitudinal | |
| 12-14 Hz | | Fasciculus | |
| Low Beta | Deficient | Corpus collosum | 500J @ 15 Hz |
| 15-17 Hz | | | C-2 (between |
| | | | Cz and C4) |
| | | | P-2 (between |
| | | | Pz and P4) |
| Middle | Deficient | Left inferior fronto-occipital fasciculus | 250J @ 20 Hz |
| Beta | | (PRIMARY) | F3 |
| 18-24 Hz | | | 250J @ 20 Hz |
| STEP 1 | | | O1 |
| | | Right inferior fronto-occipital fasciculus | 250J @ 20 Hz |
| | | (PRIMARY) | F4 |
| | | | 250J @ 20 Hz |
| | | | O2 |
| | | Right superior longitudinal | 250 J @ 20 Hz |
| | | fasciculus | F4 |
| High Beta | Deficient | Corpus collosum | 250 J F4 @ |
| 25-29 Hz | | | 25 HZ |
| 30 Hz | Severely deficient | Right Corticothalamic tract | 250 J @ 30 |
| STEP 3 | | Right Fronto-pontine tract | Hz: |
| | | Right cingulate tract | @ P3-P4 |
| | | Corpus Collosum | 500J @ 30 Hz |
| | | | @ C4 to F4 |

B. Treatment Plan

Thiamine to Support Striatum

Step 1: On Left:

250 J @ 20 Hz—FP1

250 J @ 20 Hz—O1

INCREASE JOULES SLOWLY

On Right:

250 J @ 20 Hz—FP2

250 J @ 20 Hz—O2

INCREASE JOULES SLOWLY

C. Step 2: Gamma

FP1-F3-F7—start with 250 J

P4—start with 250 J

INCREASE JOULES SLOWLY

REPEAT Q EEG—DETERMINE IF STEP 3 IS NECESSARY

D. Step 3: 30 HZ

250 J @ 30 HZ @ P3-P4

500 J @ 30 HZ @ C4 TO F4
Laser Treatment Log: Patient Name: AK
Bold Areas Reflect Changes

| | | | | If | TOTAL | |
|---|---|---|---|---|---|---|
| Date of | Area | # Joules | | pulse | Joules | |
| Treatment | Treated | delivered | Watts | # Hz | Delivered | Comments |
| Mar. 2, | FP1 | 250 J | 10 W | 20 Hz | 250 J | |
| 2020 | FP2 | | | | | |
| | O-1 | 250 J | 5 W | 20 Hz | 250 J | |
| | O-2 | | | | | |
| Mar. 4, | FP1 | 125 J | 10 W | 20 Hz | 125 J | Felt giddy |
| 2020 | FP2 | | | | | agitated but |
| Laser # 2 | | | | | | less visual |
| | | | | | | distortion |
| | | | | | | and more |
| | | | | | | logical after |
| | | | | | | the laser |
| | O-1 | 125 J | 5 W | 20 Hz | 125 J | |
| | O-2 | | | | | |
| Mar. 6, | FP1/2 | 250 J | 10 W | 20 Hz | | |
| 2020 | FP3/4 | | | | | |
| Laser # 3 | | | | | | |
| | O-1 and | 250 J | 5 W | 20 Hz | | |
| | between | | | | | |
| | O1 P3 | | | | | |
| | O-2 and | | | | | |
| | and | | | | | |
| | between | | | | | |
| | O2 and | | | | | |
| | P4 | | | | | |
| Mar. 9, | FP1/2 | 250 J | 10 W | 20 Hz | | No giddiness |
| 2020 | | | | | | after the last |
| Laser # 4 | | | | | | laser . . . |
| | | | | | | which was |
| | | | | | | spread out |
| | | | | | | over larger |
| | | | | | | area. Will re- |
| | | | | | | concentrate. |
| | O1/02 | 250 J | 5 W | 20 Hz | | |

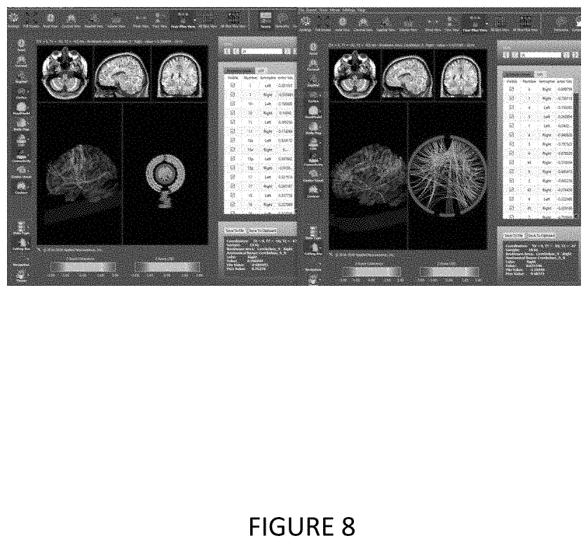

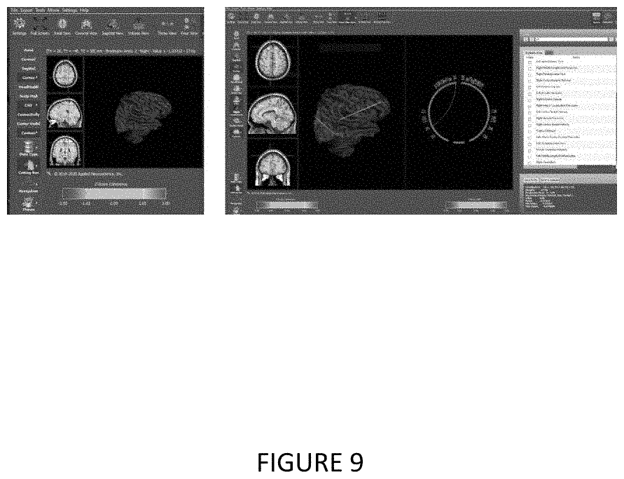

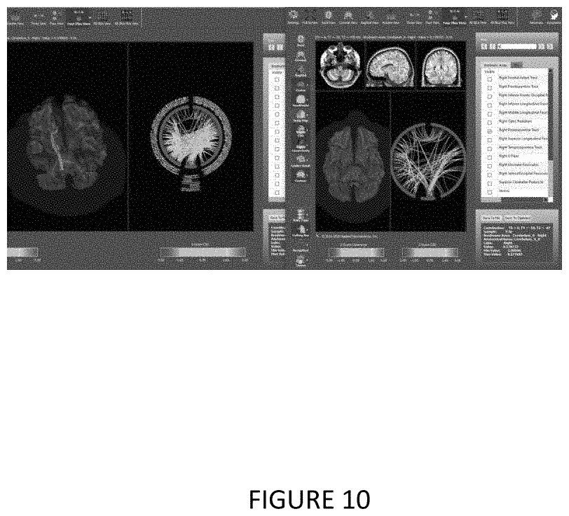

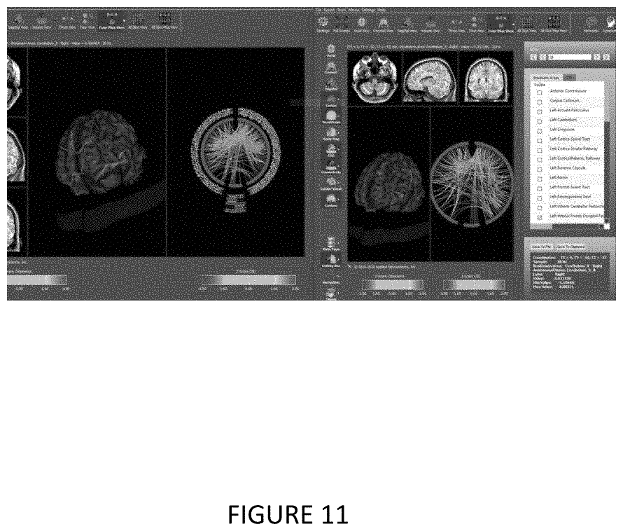

1.1.12 Follow Up qEEG:
   FIG. 8: 18 Hz: Left Inferior Fronto Occipital Fasciculus—Temporal Connection—Visual Object Recognition, Semantic Processing
   FIG. 9: 17 Hz Pre (Right) Post (Left) Vertical Occipital Fasciculus (Vision and cognition, and reading)
   FIG. 10: Pre (Left panel)—Post (right panel) qEEG: Parieto-pontine tract normalized
   FIG. 11: Normalization of Left Inferior Fronto-Occipital Fasciculus: Salience network, semantic language
1.1.13 Outcome

Objective: See qEEG results above.

CNS Vital Signs

| | Oct. 6, 2018 | Jun. 16, 2020 |
|---|---|---|
| Test | Percentile Score | Percentile Score |
| Neuro-cogntive Index | 25 | 55 |
| (Overall Score) | | |
| Composite Memory | 30 | 40 |
| Verbal Memory | 70 | 77 |
| Visual Memory | 12 | 16 |
| Psychomotor Speed | 32 | 70 |
| Reaction Time | 37 | 37 |
| Complex Attention | 9 | 86 |
| Cognitive Flexibility | 27 | 94 |
| Processing Speed | 37 | 50 |
| Executive Function | 42 | 94 |
| Reasoning | 37 | 92 |
| Simple Attention | 40 | 70 |
| Motor Speed | 30 | 77 |

Subjective:

Mar. 19, 2020: “I am more able to detect the distortions—with parents. Not measuring it.

A lot better than a month ago—“I am convinced that the visual distortions are not real, so don't have to buy into them.” It will be uncomfortable to continue having them. Frequency of visual distortions—no change Believability has gone down—they are less visually convincing—they don't look as real. I can visually see signs it is a distortion—someone's face moving in an unnatural way. See something quickly and it will morph . . . can remember moments when I just sensed menace from people/judgement/disdain from age 2-3—remember being socially phobic, afraid to talk to people unless I know them well. I think the distortions are less fleshed out, weaker.” “My reading seems to have speeded up a lot.”

Apr. 20, 2020: Not having visual distortions with family, or on Zoom; They have been gone since April 10 (finished laser Mar. 10, 2020).

May. 25, 20: “The good news is that the eight weeks between the laser therapy and missing the Luvox were the best yet.”

### HyLaNe Method Flow Sheet

Evaluation of Patient

Testing of Systems Identified as Relevant:
a) Digestion
b) Immune/Infectious/inflammatory
c) Detoxification
d) Mitochondrial
e) Endocrine
f) Genetics
g) Epigenetics
h) Structural (e.g., apnea, MRI, Sleep Study, Cardiac)
i) Quantitative Electroencephalogram (qEEG)

Review and Integration of Data

Creation of a Sequenced Treatment Plan

Treatment:
a) Urgent interventions
b) Hormonal Interventions
c) Gastrointestinal Interventions
d) Specific Nutritional Interventions
e) Detoxification Interventions
f) Brain specific treatments: Hyperbaric Oxygen Therapy (HBOT), Nimodipine (brain injury)
   Neurofeedback; Tissue High Intensity Laser

Algorithm for Use of Laser—ISNT THIS REDUNDANT?
i) Identify Cortical areas of over activity and under activity and correlate with specific symptoms
j) Identify DTI tracts (Diffuse Tensor Imaging) which are over/under active and correlate with specific symptoms
k) Determine a sequence of which areas to treat first; e.g., treat under or over active areas first
l) Determine which areas are primary (based on symptoms, degree of abnormality, location, tract/cortical function), which are secondary.
m) Determine whether to use pulse (p) or continuous wave (cw); what wavelength of infrared light (e.g., 810 nm, 1064 nM), if pulsed, what pulse frequency (Hz) to use; in which area; how many joules to deliver (ranger of 1-60 CM²); what wattage (5 W-30 W); what frequency of treatment (1/day to 1 per week).
n) This is determined by the frequency in which the cortical or DTI tract disturbance appears—i.e. if the superior longitudinal tract DTI is underactive in the alpha band, we could elect to treat with 10 HZ light.
o) Assess patient response to prior treatment before each treatment
p) Patient shaves in the areas that will be treated
q) The area(s) of treatment are marked using a 10/20 cap for location and a mascara pen for marking the scalp.
r) The plan of each treatment is written down: location, the area (in CM²) to be treated, pulse vs continuous wave (if pulsed, then frequency of pulse is chosen); wattage to be used; total # of joules to be delivered.

While the invention has been described in detail in connection with only a limited number of embodiments, it should be readily understood that the invention is not limited to the foregoing description.

### NON-PATENT CITATIONS

1. Hamblin, M R “Shining light on the head: Photobiomodulation for brain disorders,” BBA Clinical (6):113-124 (2016).

2. Henderson, T A et al. “Near-infrared photonic energy penetration: can infrared phototherapy effectively reach the human brain?” Neuropsychiatric Disease and Treatment (11):2191-2208 (2015).

3. Kraft, H H “Transcranial Laser Therapy of Post-Traumatic stress disorder in an Autistic Patient,” Psychol. Behav Sci Int J 10(2):001-004 (2019).

1. A method for a processor controlling a laser system to automatedly arrange to treat a neuropsychiatric condition in a subject comprising the steps of:
being provided results from an MRI of the subject;
being provided symptoms of the subject;
based on said symptoms and said MRI results, calculating potential target areas, tracts or networks in said subject's brain where functioning is outside a normal or desired range by administering a first test;
based on said first test, determining cortical under and/or over activity, and applicable tracts and networks to administer a laser application;
based on said determined tracts, networks and cortical activity, calculating laser parameters for application, said processor-calculated laser parameters including selection of laser wavelength, continuous or pulsed wave, frequency of administration, duration of administration, wattage, and joules to be delivered by said laser application;
determining a laser application delivery approach, said approach including processor-determined parameters and quantity and frequency of application;
directing said laser emitting system to apply said selected laser application according to said selected parameters and approach; and
administering a second test and determining quantitative differences between the results of said first and said second tests, thereby adjusting said parameters and approach as appropriate;
wherein said determined differences are further used to determine success in treating said neuropsychiatric disorder.

2. The method of claim 1, where said first test results include identifying specific areas, networks and/or tracts in the brain where functioning is outside a normal or desired range in a physical procedure.

3. The method of claim 2, where said each of said first and said second test is a qEEG.

4. The method of claim 1, where said application is directed to stimulating or inhibiting brain neurons.

5. The method of claim 1, further including the step of assessing the subject's diet and environment as inputs to calculating said parameters and approach.

6. The method of claim 1, where said symptoms include memory issues.

7. A method for a processor to deliver a calculated, targeted laser delivery approach to improve a patient's neurological condition comprising the steps of:
determining symptoms of the subject and a diagnosis at least in part by analysis of MRI and qEEG results;
based on said symptoms and diagnosis, identifying specific areas, networks and/or tracts in said subject's brain where functioning is outside a normal or desired range by administering a first test, said first test directed to brain areas selected based on said symptoms;
determining cortical tract and network under and/or over activity from results of said first test;
based on said identification and determined cortical network and/or tract activity, identifying targets for administration of laser application, said laser application applied to said targets based on processor-selected parameters including selection of laser wavelength, continuous or pulsed wave, frequency of administration, duration of administration, wattage, and joules to be delivered by said laser application;
applying selected laser applications according to said selected parameters; and
administering a second test and measuring quantitative differences between said first and said second tests, thereby adjusting said parameters for additional laser application;
wherein said measured differences are used to determine success in treating said disorder.

8. The method of claim 7, where each of said first and second test is a qEEG.

9. The method of claim 7, where said diagnosis include Parkinson's Disease.

10. The method of claim 7, where said diagnosis include Lewy Body Dementia.

11. The method of claim 7, where said diagnosis include Alzheimer's Disease.

12. The method of claim 7, where said diagnosis include seizures.

13. The method of claim 7, where said diagnosis include social phobia.

14. A method for a processor to administer targeted laser therapy to a neurological patient comprising the steps of:
identifying at least one cortical area, network, or tract in said patient's brain where functioning is outside a normal range by administering a first series of tests at least including an MRI and a qEEG;
selecting parameters for laser application based on analyzing results of said first series of tests, said parameters including selection of laser wavelength, continuous or pulsed wave, frequency of administration, duration of administration, wattage, and joules to be delivered by said laser application;
applying a series of laser applications according to said selected parameters at selected time intervals;
administering a second test and calculating quantitative neurological differences between the results of said first series of tests and said second test; and
applying an additional series of laser applications where said parameters are adjusted based on result differences from said first test to said second test.

15. The method of claim 14, where said determination includes identifying at least one specific cortical area, network, or tract in the brain where functioning is outside a normal range, determined in a physical procedure.

16. The method of claim 15, where said second test includes at least a qEEG.

17. The method of claim 15, where at least 10 laser applications are applied.

18. The method of claim 15, where the number of laser applications is at least 20 before said second test.

19. The method of claim 15, where the patient is clinically tested during each laser application and laser parameters may be adjusted accordingly.

20. The method of claim 15, where said laser applications are directed to at least one of theta, beta, and alpha improvement.
